(19) United States
(12) Patent Application Publication
(10) Pub. No.: US 2026/0043034 A1
(43) Pub. Date: Feb. 12, 2026

(54) RECOMBINANT ENGINEERING BACTERIUM FOR IMPROVING YIELD OF RECOMBINANT HUMAN ALBUMIN

(71) Applicant: TONGHUA ANRATE BIOPHARMACEUTICAL CO., LTD., Tonghua (CN)

(72) Inventors: Wei XIANG, Tonghua (CN); Guoli GONG, Tonghua (CN); Xu HAN, Tonghua (CN); Ning WANG, Tonghua (CN); Xiufeng LI, Tonghua (CN)

(73) Assignee: TONGHUA ANRATE BIOPHARMACEUTICAL CO., LTD., Tonghua (CN)

(21) Appl. No.: 19/234,290

(22) Filed: Jun. 11, 2025

(30) Foreign Application Priority Data
Aug. 9, 2024 (CN) 202411087607.8

(51) Int. Cl. C12N 15/81; C07K 14/76; C12N 9/22; C12N 15/11; C12N 15/90

(52) U.S. Cl. CPC C12N 15/815; C07K 14/76; C12N 9/226; C12N 15/11; C12N 15/905; C12N 2310/20

(57) ABSTRACT

A recombinant engineering bacterium for improving a yield of a recombinant human albumin is provided, which relates to the field of biological chemistry, particularly to the fields of gene engineering technologies and microorganisms. Targeted knockout is conducted on one or more genes of CCW14 gene, EMW1 gene, and PUN1 gene by utilizing a CRISPR-Cas9 gene editing technology, and/or one or more genes of EXG1 gene and SPR1 gene are over-expressed and integrated into a strain of the recombinant engineering bacterium, so as to improve the expression level of the recombinant human albumin.

### CROSS-REFERENCE TO RELATED APPLICATIONS

This application claims priority to Chinese Patent Application No. 202411087607.8, filed on Aug. 9, 2024, the contents of which are hereby incorporated by reference to its entirety.

### SEQUENCE LISTING

The instant application contains a Sequence Listing which is submitted electronically in XML format and is hereby incorporated by reference in its entirety. The XML copy, created on Oct. 31, 2025, is named “2025 Oct. 31-Sequence List-63601-H001US00”, and is 52,524 bytes in size.

### TECHNICAL FIELD

The present disclosure relates to the fields of gene engineering technologies and microorganisms, particularly to a recombinant engineering bacterium for improving the yield of a recombinant human albumin.

### BACKGROUND

As a model eukaryote, yeast is simple in genetic operation, relatively complete in protein post-translational modification and secretion pathways, rapid in growth speed and low in nutrition requirements, and is suitable for large-scale high-density fermentation. It is found by studies on secretion expression of a large amount of different heterologous proteins in the yeast that the expression levels of different proteins vary greatly. Some heterologous proteins have a limited ability in secretion, thereby limiting the production efficiency of proteins. The improvement of a key gene for biosynthesis and secretion mechanisms of a protein through gene manipulation is a major means for improving the production efficiency of the heterologous protein.

As an important component of a yeast cell and a barrier for isolating cells from the outside world, a cell wall is capable of maintaining cell morphology, and plays a crucial role in cell growth, division, conditioned stress and cell fusion mating and maintenance of cell integrity, etc.

A yeast cell wall is mainly composed of mannoprotein, β-glucan, and a small amount of chitin. The β-glucan is a multi-branch polymer, with its main chain being linked by a β-1,3-glycosidic bond and branched by a β-1,6-glycosidic bond, so as to form a network structure. The chitin is formed by polymerizing N-acetylglucosamine through a β-1,4-glycosidic bond. The mannoprotein is first linked to β-1,6 glucan of the cell wall through a covalent bond and then linked to β-1,3 glucan through β-1,6 glucan, or directly linked to β-1,3 glucan. The chitin is also linked to a non-reduced terminal of a β-1,3-glucan chain through the β-1,4-glycosidic bond at a reduced terminal of the chitin, or indirectly linked to a β-1,6 glucan chain by using the β-1,3-glucan chain as a side chain.

Many studies have proved that changes in components and a structure of the cell wall may affect protein secretion. A cell wall protein gene CWP2 disrupting strain is constructed by using recombinant Saccharomyces cerevisiae for producing heterologous β-glucosidase as a starting strain. After fermentation for 96 h, the enzyme activity of extracellular β-glucosidase is improved by 53%, the intracellular enzyme activity is improved by 208%. Through the knockout of a cell wall-related protein gene DFG5 of Saccharomyces cerevisiae, the secretion expression activity of heterologous protein β-glucosidase is improved by 3 times. Through the knockout of a cell wall synthesis-related gene KIGAS1-1 in Kluyveromyces lactis, it has found that the thickness of the cell wall is reduced, and the secretion amount of exogenous protein xylanase B is improved. The above cases provide good examples for constructing the recombinant engineering bacterium highly expressing the recombinant human albumin.

The recombinant engineering bacterium of the present disclosure can significantly improve the yield of the recombinant human albumin by up to 238% and has wide application prospects.

### SUMMARY

One or more embodiments of the present disclosure provide a recombinant engineering bacterium for improving a yield of a recombinant human albumin. The recombinant engineering bacterium comprises, or is prepared with, the following operations while expressing the recombinant human albumin: conducting targeted knockout on one or more genes of CCW14 gene, EMW1 gene, and PUN1 gene by utilizing a CRISPR-Cas9 gene editing technology; and over-expressing and integrating one or more genes of EXG1 gene and SPR1 gene into a strain of the recombinant engineering bacterium. In essence, for one or embodiments of the present disclosure, the recombinant engineering bacterium is prepared by the operations noted above, performed to modify, in some cases, Komagataella phaffii. A nucleotide sequence of the SPR1 gene is as shown in SEQ ID NO.7, a nucleotide sequence of the EXG1 gene is as shown in SEQ ID NO.9, and the recombinant engineering bacterium comprises at least one copy of a nucleotide sequence encoding the recombinant human albumin, and the recombinant engineering bacterium is formed by modifying Komagataella phaffii.

One or more embodiments of the present disclosure provide a method for preparing a recombinant human albumin. The method comprises: making the recombinant human albumin by fermenting the above recombinant engineering bacterium.

### BRIEF DESCRIPTION OF THE DRAWINGS

Drawings described herein are used for further understanding the present disclosure and constitute one part of the present disclosure. Exemplary embodiments and descriptions thereof of the present disclosure are used for explaining the present of the present disclosure, and do not constitute an improper limitation of the present of the present disclosure. In the drawings:

FIG. 1 is a structural diagram of a recombinant expression cassette pPICZA-rHA encoding a recombinant human albumin;

FIG. 2 is a structural diagram of a recombinant expression cassette pART202 encoding a Cas9 recombinant nucleic acid molecule and a CCW14-gRNA recombinant nucleic acid molecule;

FIG. 3 is a structural diagram of a recombinant expression cassette pART204 encoding a Cas9 recombinant nucleic acid molecule and an EMW1-gRNA recombinant nucleic acid molecule;

FIG. 4 is a structural diagram of a recombinant expression cassette pART206 encoding a Cas9 recombinant nucleic acid molecule and a PUN1-gRNA recombinant nucleic acid molecule;

FIG. 5 is a structural diagram of a recombinant expression cassette pART210 encoding an SPR1 recombinant nucleic acid molecule and an EXG1 recombinant nucleic acid molecule;

FIG. 6 is a result graph of a standard curve of an HIS4 gene in example 1;

FIG. 7 is a result graph of a standard curve of an rHA gene in example 1;

FIG. 8 is a colony atlas in example 4;

FIG. 9 is a colony atlas in example 5;

FIG. 10 is a sodium dodecyl sulfate-polyacrylamide gel electrophoresis (SDS-PAGE) electropherogram in example 6, in the figure, electropherograms numbered 1-9 are electropherograms showing the expression of recombinant human albumins in recombinant engineering bacteria 1-9, the electropherogram numbered 10 is an electropherogram of a human serum albumin, and the electropherogram numbered 11 is a protein Marker;

FIG. 11 is a result diagram showing that recombinant engineering bacteria express the recombinant human albumin in example 6.

### DETAILED DESCRIPTION OF THE EMBODIMENTS

The relevant terms that appear in embodiments are illustrated as follows.

The expression cassette refers to a gene expression system including all necessary elements required by exogenous protein expression, including promoters, exogenous gene cloning sites, signal peptide sequences, mature peptide coding sequences of target proteins, terminators, screening markers, etc. The target proteins are proteins that need to be specifically obtained, studied, or applied.

The vector refers to autonomous DNA capable of introducing exogenous DNA into a host cell for replication or finally making exogenous gene DNA expressed. The vector mainly includes a cloning vector and an expression vector, the cloning vector is used for replication and amplification of genes, and the expression vector is used for expression of target genes. The target genes are genes need to be specifically expressed.

The host cell refers to a cell receiving exogenous genes during transformation or transduction.

The recombinant engineering bacterium refers to a fungi cell strain system for efficient expression of exogenous genes by using a genetic engineering method.

To more clearly illustrate the overall concept of the present disclosure, the present disclosure will be described in detail below in a mode of embodiments in conjunction with drawings in the disclosure. The descriptions hereinafter give a large number of specific details so as to provide a more thorough understanding of the present disclosure. However, it is obvious for those skilled in the art that the present disclosure will be implemented without one or more of these details. In other examples, to avoid confusion with the present disclosure, some well-known technical features in the art have not been described.

If specific conditions are not specified in the embodiments, conventional conditions or conditions recommended by a manufacturer are employed.

Unless otherwise specified, in the following embodiments, reagents or instruments used that do not indicate manufacturers are all conventional products that are commercially available.

Molecular biology experimental methods that are not specifically described in the following examples are all carried out according to kit and product instructions, or by reference to methods described in a book entitled “Molecular Cloning Experiment Guidelines” (Third Edition) written by J. Sambrook.

In some embodiments, the source strain of the recombinant engineering bacterium is Komagataella phaffii. In some embodiments, the source strain of the recombinant engineering bacterium includes Pasteur Komagataella phaffii CBS7435 purchased from American Type Culture Collection (ATCC), which is also called Komagataella phaffii NRRL Y-11430, with ATCC Number: 76273. In some embodiments, the recombinant engineering bacterium is constructed by gene editing of the source strain. In some examples, the recombinant engineering bacterium is constructed by gene knockout and/or gene overexpression of the source strain. The specific construction method is detailed elsewhere in this disclosure.

In some embodiments, the expression vector includes at least one of an expression vector pPICZA (a specification of 20 μL, purchased from Hunan Fenghui Biotechnology Co., Ltd.), an expression vector pGAPZA (a specification of 20 μL, purchased from Hunan Fenghui Biotechnology Co., Ltd.), etc.

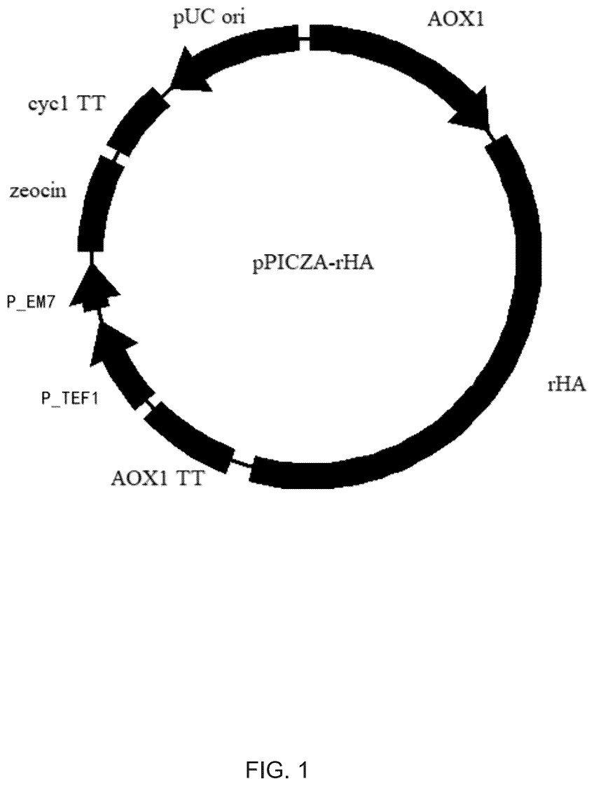

FIG. 1 is a structural diagram of a recombinant expression cassette pPICZA-rHA encoding a recombinant human albumin.

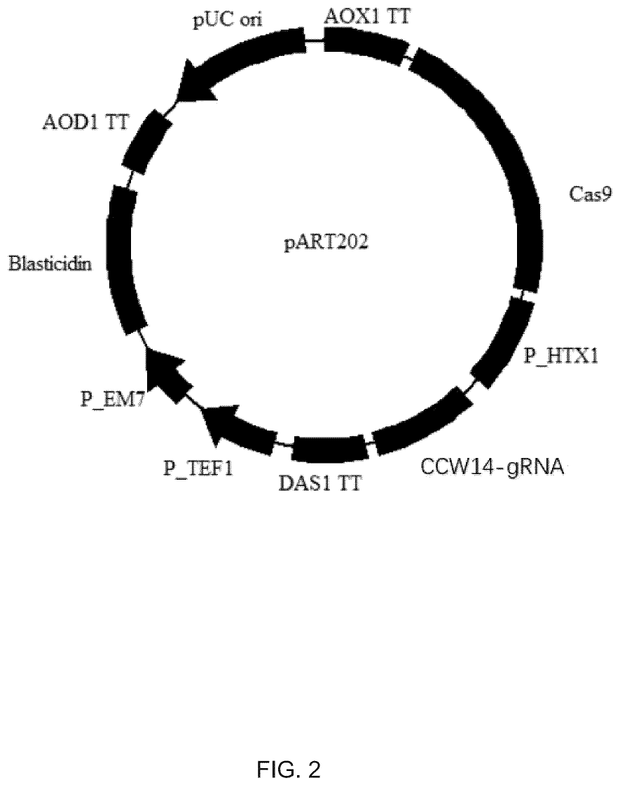

FIG. 2 is a structural diagram of a recombinant expression cassette pART202 encoding a Cas9 recombinant nucleic acid molecule and a CCW14-gRNA recombinant nucleic acid molecule.

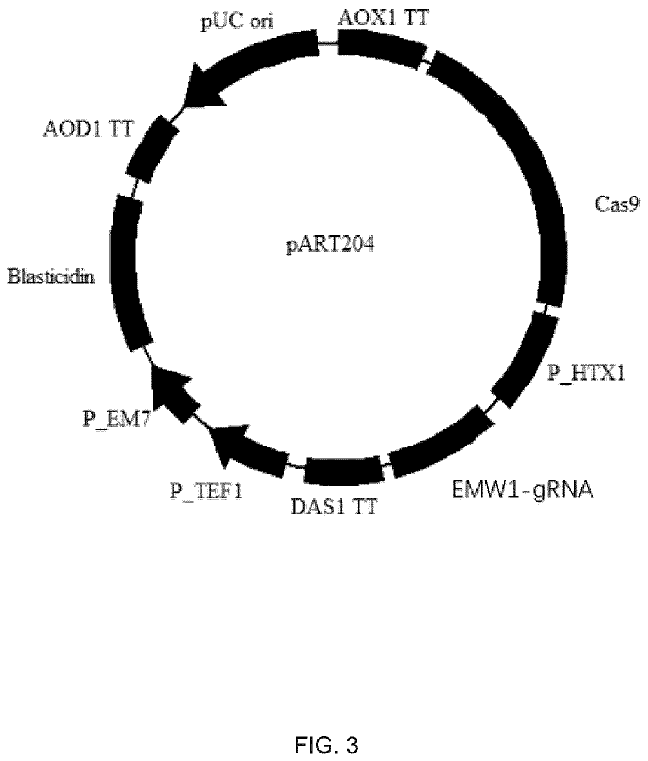

FIG. 3 is a structural diagram of a recombinant expression cassette pART204 encoding a Cas9 recombinant nucleic acid molecule and an EMW1-gRNA recombinant nucleic acid molecule.

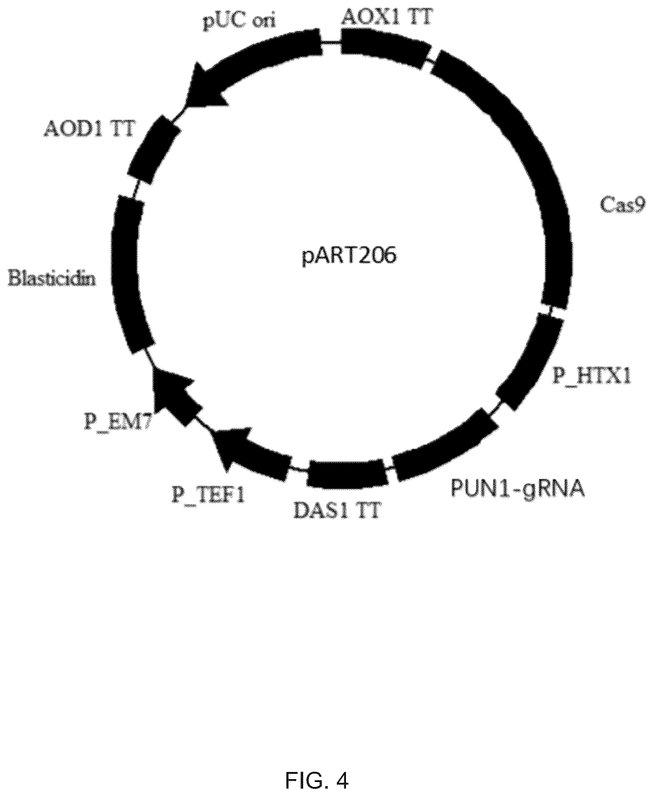

FIG. 4 is a structural diagram of a recombinant expression cassette pART206 encoding a Cas9 recombinant nucleic acid molecule and a PUN1-gRNA recombinant nucleic acid molecule.

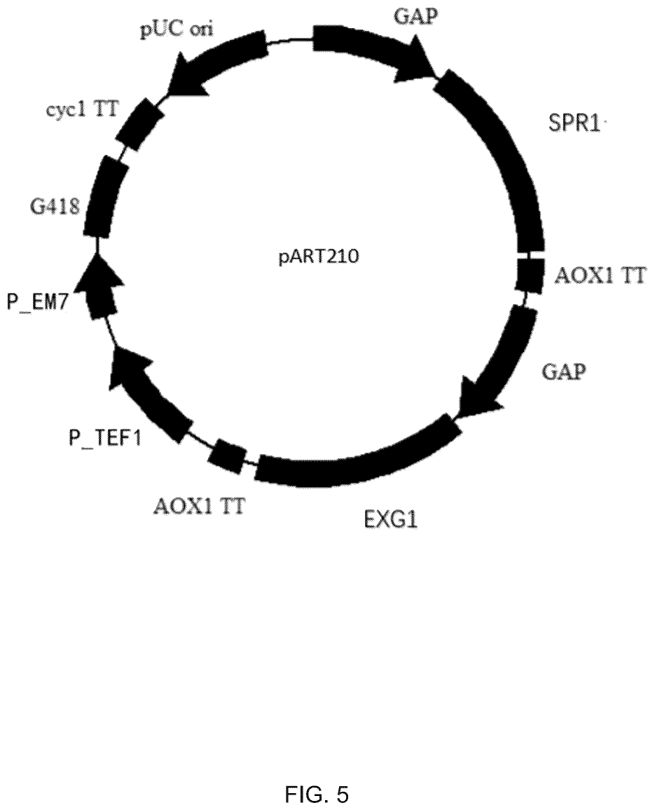

FIG. 5 is a structural diagram of a recombinant expression cassette pART210 encoding an SPR1 recombinant nucleic acid molecule and an EXG1 recombinant nucleic acid molecule.

In some embodiments, the following expression cassettes are constructed in a lab.

The expression cassette pPICZA-rHA may encode a recombinant nucleic acid molecule of at least one recombinant human albumin, the structural diagram of which is as shown in FIG. 1.

The expression cassette pART202 may encode a Cas9 recombinant nucleic acid molecule and a CCW14-gRNA recombinant nucleic acid molecule, the structural diagram of which is as shown in FIG. 2.

The expression cassette pART204 may encode a Cas9 recombinant nucleic acid molecule and an EMW1-gRNA recombinant nucleic acid molecule, the structural diagram of which is as shown in FIG. 3.

The expression cassette pART206 may encode a Cas9 recombinant nucleic acid molecule and a PUN1-gRNA recombinant nucleic acid molecule, the structural diagram of which is as shown in FIG. 4.

The expression cassette pART210 may encode an SPR1 recombinant nucleic acid molecule and an EXG1 recombinant nucleic acid molecule, the structural diagram of which is as shown in FIG. 5.

In some embodiments, the enzyme used in the process of constructing the recombinant engineering bacterium includes a restriction endonuclease BglII (a specification of 2000 units, purchased from NEB) and a PrimeSTAR high fidelity DNA polymerase (a specification of 100 Rxns, purchased from Takara), etc.

In some embodiments, the kit used in the process of constructing the recombinant engineering bacterium includes at least one of a plasmid extraction kit (a specification of 50 preps), an agarose gel extraction kit (a specification of 50 preps), a DNA product purification kit (a specification of 50 preps), a yeast genomic DNA extraction kit (a specification of 50 preps), etc., which are purchased from Tiangen Biotechnology Co., Ltd.

In some embodiments, the antibiotic used in the process of constructing the recombinant engineering bacterium includes at least one of Zeocin (a specification of 10 mL), Geneticin (a specification of 20 mL), Blasticidin (a specification of 20 mL), etc., which are purchased from Thermo Fisher Scientific.

In some embodiments, the culture medium used in the process of constructing the recombinant engineering bacterium includes Escherichia coli culture medium LB (1% (w/v) tryptone, 0.5% (w/v) yeast extract, 1% (w/v) NaCl, and a pH of 7.0), yeast culture medium YPD (1% (w/v)) yeast extract, 2% (w/v) peptone, and 2% (w/v) glucose), etc.

In some embodiments, the culture medium further includes a yeast screening culture medium YPDZ (YPD+different concentrations of Zeocin), a yeast screening culture medium YPDG (YPD+different concentrations of Geneticin), a yeast screening culture medium YPDB (YPD+different concentrations of Blasticidin), etc.

In some embodiments, the culture medium further includes a yeast culture medium BMGY (1% (w/v)) yeast extract, 2% (w/v) peptone, 1.34% (w/v) YNB, 0.00004% (w/v) Biotin, and 1% glycerinum (V/V)), a yeast induced culture medium BMMY (1% (w/v)) yeast extract, 2% (w/v) peptone, 1.34% (w/v) YNB, 0.00004% (w/v) Biotin, and 0.4% methanol (V/V)), etc. YNB is yeast nitrogen base.

In some embodiments, an amino acid sequence of the recombinant human albumin is as shown in SEQ ID NO.11 (DAHKSEVAHRFKDLGEENFKALVLIAFAQYLQQCPFEDHVKLVNEVTEFAKTCVADESA ENCDKSLHTLFGDKLCTVATLRETYGEMADCCAKQEPERNECFLQHKDDNPNLPRLVRP EVDVMCTAFHDNEETFLKKYLYEIARRHPYFYAPELLFFAKRYKAAFTECCQAADKAAC LLPKLDELRDEGKASSAKQRLKCASLQKFGERAFKAWAVARLSQRFPKAEFAEVSKLVT DLTKVHTECCHGDLLECADDRADLAKYICENQDSISSKLKECCEKPLLEKSHCIAEVEND EMPADLPSLAADFVESKDVCKNYAEAKDVFLGMFLYEYARRHPDYSVVLLLRLAKTYE TTLEKCCAAADPHECYAKVFDEFKPLVEEPQNLIKQNCELFEQLGEYKFQNALLVRYTK KVPQVSTPTLVEVSRNLGKVGSKCCKHPEAKRMPCAEDYLSVVLNQLCVLHEKTPVSD RVTKCCTESLVNRRPCFSALEVDETYVPKEFNAETFTFHADICTLSEKERQIKKQTALVEL VKHKPKATKEQLKAVMDDFAAFVEKCCKADDKETCFAEEGKKLVAASQAALGL). A nucleotide sequence of the recombinant human albumin is as shown in SEQ ID NO.12 (gacgctcacaagtctgaagttgcccacagattcaaggacctgggtgaagaaaacttcaaggccctggttttgatcgctttcgctcaatacttg cagcagtgtccattcgaggaccacgttaagttggttaacgaggttactgagttcgccaagacttgtgttgctgacgaatctgctgagaactgc gataagtccttgcacactttgttcggtgacaagttgtgtaccgttgccaccttgagagaaacctacggtgaaatggctgactgttgtgctaagc aagagccagaaagaaacgagtgcttcttgcagcacaaggacgacaacccaaacttgccaagattggttagaccagaggttgacgttatgtg tactgccttccacgacaacgaggaaaccttcttgaagaagtacctgtacgagatcgctagaaggcacccatacttttacgctccagagttgtt gttcttcgccaagcgttacaaggctgctttcactgagtgttgtcaagctgctgacaaggccgcttgtttgttgccaaagttggacgagttgcgtg acgaaggtaaggcttcttccgctaagcagagattgaagtgtgcctccttgcaaaagttcggtgagagagcttttaaggcttgggctgttgcta gactgtcccagagatttccaaaggctgagtttgctgaggtttccaagttggtcactgacttgactaaggttcacaccgaatgttgccacggtga cttgttggaatgtgctgatgatagagccgacctggccaagtacatttgtgaaaaccaggactccatctcctccaagttgaaagagtgttgcga gaagccactgttggagaagtctcactgtatcgctgaagttgagaacgacgaaatgccagctgacttgccatctttggctgctgacttcgttga atccaaggacgtctgtaagaactacgctgaagctaaggacgtgttcctgggtatgttcttgtacgagtacgctcgtagacacccagactactc cgttgtcttgttgttgagattggccaagacctacgagactaccttggaaaagtgttgtgctgctgctgacccacacgaatgttacgctaaggttt tcgacgagttcaagccattggttgaggaaccacagaacctgatcaagcagaactgtgagttgttcgagcagctgggtgagtacaagttcca gaacgctttgttggtcagatacaccaagaaggtcccacaggtttccactcctaccttggttgaagtctccagaaaccttggtaaggtcggttcc aagtgttgtaagcacccagaggctaagagaatgccatgtgctgaagattacttgtccgtcgtcttgaaccagttgtgcgtcttgcacgaaaag actccagtttccgacagagttaccaagtgctgtactgagtccttggtcaaccgtagaccatgtttctctgctttggaagtcgacgagacttacgt cccaaaagagttcaacgctgagactttcactttccacgctgacatctgtaccctgtccgaaaaagagagacagatcaagaagcagactgcct tggtcgaattggtcaagcacaagccaaaggctaccaaagagcagttgaaggctgttatggatgacttcgctgctttcgttgagaagtgttgca aggctgacgacaaagagacttgtttcgctgaagagggtaagaagttggttgctgcttcccaagctgctttgggtctgtaa) or as shown in a nucleotide sequence at least having 98% identity to SEQ ID NO.12.

The recombinant engineering bacterium constructed by some embodiments of the present disclosure are used to express the recombinant human albumin.

In some embodiments, the construction method of the recombinant engineering bacterium includes conducting targeted knockout on one or more genes of CCW14 gene, EMW1 gene, and PUN1 gene of the source strain by utilizing the CRISPR-Cas9 gene editing technology. The construction method of the recombinant engineering bacterium includes the following steps:
S1: constructing a Cas9 and gRNA co-expression plasmid.
S2: preparing donor DNA.
S3: preparing a competent cell.
S4: constructing a strain with one or more genes of CCW14 gene, EMW1 gene, and PUN1 gene being knocked out (i.e., the recombinant engineering bacterium).

In some embodiments, the recombinant engineering bacterium with one or more genes of CCW14 gene, EMW1 gene, and PUN1 gene being knocked out is obtained by transferring the Cas9 and gRNA co-expression plasmid and the donor DNA into the competent cell of the source strain.

In some embodiments, an amino acid sequence encoded by the CCW14 gene is as shown in SEQ ID NO.2 (MQFTFASTSVVVSLIAALAKPAVATPPACLLACAAEVVKESSDCDALNNIQCICENEGSAI HACLESTCPDGLSSTALQSFEDVCESVGTEANLDESSSSQSSSSSSSSESSSSSVSSSSSSAS SSSETSSSVTSSSVTSSSTAVSSSTESSSSVEPSTSHSSSHSSSEVSSTVAPTTSVAPTTSSITTS STSLTSATTSSVTISIEPTSDAADKVIIPGLAGLVGALAVGLI). A nucleotide sequence of the CCW14 gene is a nucleotide sequence encoding a protein with a sequence shown in SEQ ID NO.2. In some embodiments, the nucleotide sequence of the CCW14 gene is as shown in SEQ ID NO.1 (atgcagtttactttcgcttctacctctgttgttgtctcactgattgctgctctcgctaagcctgctgttgccactccaccggcttgcctgttggcct gtgctgccgaggttgttaaggaatcatccgactgtgatgccttgaacaacattcaatgtatctgtgagaatgaaggatcagccattcatgcctg cttggagtcaacctgtcccgatggattgtcgtccactgctttgcagtcttttgaagatgtttgcgagagtgtcggcactgaagctaaccttgatg agagctcttcatctcagtcctcatcatcgtcttcttctteggagtcatcatcgtcttccgtgtcatcatcatcctcttccgcatcttcttcttccgagac ttcctcttccgtgacttcctcttccgtgacttcctcttccactgcagtctcttccagcaccgaatcctcttcatccgttgagccategacttcccact cttcttcccactcttcgtctgaagtctcctcaactgtcgcaccaaccaccagtgttgcaccaactaccagttccatcactacttccagtacttcttt gacatcagctactacttccagtgtgactatttctatcgagccaactagcgacgctgctgacaaggttatcatcccaggtttggctggtctagtcg gtgctttggctgttggtctgatctag), and a gRNA sequence of the CCW14 gene is as shown in SEQ ID NO.13 (ggtgctggaagagactgcag).

In some embodiments, an amino acid sequence encoded by the EMW1 gene is as shown in SEQ ID NO.4 (MSALRSIHSSFIRLNIELNVGVEGFNEQTEAISLISKGQYLKVLNLPLFKKIFTDNSEIKHII AKYTQQSSASALVVLDLVNHIIMQLSKNLESLSPEVVKTTVLILGVIFLQLFLQENFTGPSI PFENEKLPLGILRDLLKLNKEASTHILSVGGETAYELMESPILLVLSVKILEEVSGSSVSVL SKDFIDMDSETFISKCNIVTNPDVVQSSIQWWKSRAYMVFASVLTGEVSLLTLLPIKLLTP NIVSTLIDLQNKNSPENQEILVLYQLESASVNFSAGVDNSVKDALAKAKTASGLQLILTGC KAKRTKFQQTGNHALLVLAKSQDEFLRSTSSDELSCESPEELQLNDEVLLERPYYEPLEE ENTLESEEKDSKRVKIDFSFEPILGSEEDSEVKLLPIALKDDEIPPVLKSLDPNNQPQLASL DMAQLLMRLIFIRENTPSNSAMVESELMALVQRITNSKNVNWTIFSKSLWERSLLEASKS KTIERGVLQMQVLVEDLGMNSVKTRMFSQSGDEQKFLKALIDPDDFQVTPNAARLKYIF QLPLTPRWALDSLLAEKLMSIGVIKSALQIYERLNLSVDAALCYALTGDEAQAEKILVERI AAHPSDARALSVLGDVKQSPELWLKSWEVGKYSKAKVSLGKYYYQPPKDSGLTKDLPA AIGHIFDALTRNPVDFETWFFYGCMGIEASNWNLAAEAFTRCVSIDNSSPQSWSNLASAL IKLGKNKEAFSALKSAIRTSQDKKVSSKIWDNFLIVAAKLSDWTSVLLASRELLTLRSNSK SSEEIVDLPVVEKLVQVLLESPYPGSEDSMDYFQKSCVDFICTMLPQVINNSDRSWRIVSR VELWRKRPWMALECNEKAYRAAVNNPELEFNETAWKTAVDACADLVAAFESLGELPGK HGADDLVCKDWKYKAKMTVRSLMSKGKSAWEDSEGWNVLQELKNSLMS). A nucleotide sequence of the EMW1 gene is a nucleotide sequence encoding a protein with a sequence shown in SEQ ID NO.4. In some embodiments, the nucleotide sequence of the EMW1 gene is as shown in SEQ ID NO.3 (atgtcagccctaaggtctattcatagctcgtttattaggctgaatatagaattgaatgtgggggttgaaggctttaacgagcaaactgaggcc atctccttaataagcaaaggtcaatatcttaaagtcctgaatttgcctcttttcaaaaagatcttcactgataattccgaaatcaagcatataattgc aaagtatacccagcaatcatcggcatcggcactagtggttttggatttagtcaatcatatcattatgcaactatccaagaatctggagtcgttga gtccagaagtagtcaaaactactgtactgattttaggtgtgatctttttacaactatttctgcaagaaaacttcactggtccttcaataccttttgag aatgaaaaattaccgctggggatattaagagacttgctgaaattgaataaagaagcctctacgcatatattgtctgttggaggtgagacagctt atgaactcatggagtctcctattctactggttctttcagtgaaaatattagaagaagtgtcaggttctagtgtcagtgttctgtccaaagatttcatt gatatggatagtgaaacgtttatttccaaatgtaatatcgttaccaatcccgatgttgtgcaatcctcgatacaatggtggaagtcaagagcttat atggtgtttgcatcggttctcactggagaagtatccctgttgaccttactgcccatcaaactgcttacaccaaacattgtttcaacattgatagattt acaaaataagaactcaccagaaaaccaggagattctggttctatatcaactggagtctgcatctgtaaatttttcagctggtgtcgacaattcgg ttaaggatgcgctggcaaaggcaaaaaccgcttctggattacagctgattctcactggctgtaaggcaaaaagaacgaagttccaacaaac cggaaaccatgctctgctagttcttgctaagagtcaagatgagttcttaagatcaactagtagcgatgaactttcttgtgaaagtcctgaggaat tgcaacttaatgacgaggtcttgttagaaaggccttactacgagccattggaggaagagaatacactagaatcagaagaaaaagactctaaa agagtaaagatagatttttcttttgagccaattcttgggtccgaagaagattcagaggttaaactgttacctattgcattgaaagatgatgaaattc ctccagtactgaagagtttggaccccaataatcaaccacaactagcatcactggatatggcacagcttttaatgagactaatcttcatcagaga aaatacaccctctaacagcgccatggttgaatctgaattgatggccttagttcagagaatcacgaactctaaaaatgtgaactggacaatatttt cgaagagtttgtgggaaagatctttgctggaagctagtaaatcaaagactattgaaagaggcgtactgcaaatgcaggtactggttgaagat cttggaatgaatagtgtcaagaccagaatgttcagtcaatcgggagacgaacagaaatttcttaaagctctgattgatccagatgactttcaag ttactcccaatgctgcgagattgaaatacatttttcagctgccgttgactccaaggtgggccctagactcacttttggctgaaaagttgatgtcaa ttggagtaattaagtcggctttacagatatatgaaaggctgaatctttcggtggatgctgctttgtgctatgctttaacaggtgatgaagctcagg ctgaaaaaattcttgtagagcgtatagctgcccatccatccgatgcccgtgcactttcggtgttaggagatgtgaaacaatctcctgagctctg gttaaagagttgggaggttggaaagtattctaaagccaaagtttctcttggaaaatactattatcaacctcctaaggattccggtttaacaaagg atttgccagctgctattggccacatttttgacgcattgacccgtaatccagttgattttgaaacttggtttttctatggttgcatgggaattgaagca tctaactggaacctcgctgctgaggcattcacaagatgtgtatccatagacaattcaagccctcaatcatggtcaaatcttgcttctgcgttaatc aaacttggtaagaataaagaagctttcagtgcattgaagagtgcaatccgaactagccaagacaagaaggtttcttcaaaaatctgggacaat ttcttgatcgtggccgccaaattgagcgattggacttctgtcttactagcctcaagggaacttttaaccttgagaagcaattcgaaatccagtga agaaattgtggatctccccgtagttgaaaagttggttcaagtactactggaatcaccctatccaggttcagaggattctatggattactttcaaaa atcgtgcgttgacttcatatgtactatgttgccacaagttatcaacaactctgacagatcttggagaattgtttctagagttgaactttggagaaag agaccttggatggcacttgagtgcaatgaaaaggcatacagggctgcggtgaacaatcctgaacttgagttcaatgaaactgcgtggaaga ctgctgttgatgcgtgtgctgacttggtagctgcttttgaatcattgggagagttacctggcaaacatggagccgacgatcttgtttgtaaagatt ggaagtataaagctaaaatgacagttcggtccctcatgtcaaagggaaaatctgcctgggaagattcagaaggctggaatgtgctacaaga gctaaaaaacagtctaatgagttaa), and a gRNA sequence of the EMW1 gene is as shown in SEQ ID NO.14 (gatgggcagtaaggtcaaca).

In some embodiments, an amino acid sequence encoded by the PUN1 gene is as shown in SEQ ID NO.6 (MGCFRFCLVIIPALLSVVACLFAIFSCIGSTKNNEFLTSIYLLEIDASNISISALIPAAANIDIS PQQLGLSDVYTLGMWGYCEGTASSDAQTDRDEILGLDNVDFTACSSPKAMYVFDPESFI QDVLNTSASLNQVNSYLDILGVDNVDISLPQDLVDYEDTIRAVSKMIFICTIIGIVLTFIQVL FSIGAFFSKGWSCATTVVSILSFVSLIIGAGGATGMYRIVQTIFNDNLGEYGVRAGLSRNF LVFYWLAVALNLIALVFWIFSICCGSTRKRSRSYTDSAEKQPMMVYQPYEQAPQHVYK). A nucleotide sequence of the PUN1 gene is a nucleotide sequence encoding a protein with a sequence shown in SEQ ID NO.6. In some embodiments, the nucleotide sequence of the PUN1 in SEQ gene is as shown ID NO.5 (atgggttgctttagattttgtctggtcataataccagccttactgtcggttgtcgcatgtttatttgcaattttctcctgtatoggatcaaccaaaaat aacgagtttttgacgtctatttaccttttggaaattgatgcctccaatatatcaattteggctctgatccctgctgccgccaatatagacatctctcct caacaattgggtttgtcagatgtctacactcttggtatgtggggatactgcgagggtacagcttcctctgatgctcaaactgacagagatgaaa ttcttggtctggacaatgttgatttcaccgcttgttcaagtccaaaggcaatgtacgtttttgatcccgaaagtttcatacaggatgtgctgaatac ctcggcatcactaaatcaagtgaactcatacctggacattcttggagtagacaacgttgacatctctcttcctcaggatttggtcgactacgagg acacaataagagcagtatccaagatgatctttatttgcaccataatcggtattgtgcttacattcatacaggttctcttctccattggagctttcttca gcaagggatggtcttgtgccaccactgttgtaagtatattgtccttcgtaagtcttatcattggtgctggtggtgcgactgggatgtaccgaattg tgcaaactattttcaatgataacctcggtgagtatggagtcagagcaggtctttctagaaactttttggtcttctactggcttgcggttgcattaaa cctgatagccttagtattttggatcttttcaatctgctgtggatctactaggaagcgtagtaggtcgtacaccgattccgcagagaagcagccg atgatggtctatcaaccgtatgaacaggctccacagcacgtatacaaatag), and a gRNA sequence of the PUN1 gene is as shown in SEQ ID NO.15 (ggtatgtggggatactgcga).

In some embodiments, a nucleotide sequence of the Cas9 gene is as shown in SEQ ID NO.16 (atggacaagaagtactccatcggtctggacatcggaactaactctgttggttgggctgttatcaccgacgagtacaaggttccatccaagaa gttcaaggtcctgggtaacactgacagacactccatcaagaagaacttgatcggagccttgttgttcgactctggtgaaactgctgaggccac cagattgaagagaactgccagaagaagatacaccagacgtaagaacagaatctgctacctgcaagagattttctccaacgagatggccaa ggtcgacgactcatttttccacagattggaagagtccttcctggtcgaagaggataagaagcacgagagacacccaatcttcggtaacatcg ttgacgaggttgcttaccacgagaagtacccaactatctaccacctgagaaagaagttggttgactccactgacaaggccgacttgagattg atctacttggctttggcccacatgattaagttcagaggtcacttcttgatcgagggtgacttgaacccagacaactctgacgttgacaagctgtt catccagttggtccagacctacaaccagctgttcgaagagaaccctattaacgcttctggtgttgacgctaaggctatcttgtctgccagattgt ccaagtccagaagattggagaacctgatcgctcaattgccaggtgagaagaagaacggtttgttcggtaacttgattgccctgtccttgggttt gaccccaaacttcaagtccaacttcgatttggctgaggacgccaagttgcagttgtctaaggatacttacgacgacgacctggacaacttgtt ggctcaaattggtgaccagtacgccgacttgtttttggctgctaagaacttgtccgacgccatcttgttgtccgacatcttgagagttaacaccg agatcactaaggctccattgtccgcttccatgatcaagagatacgacgaacaccaccaggacttgactttgttgaaggccttggttagacagc agctgcctgagaagtacaaagaaatcttcttcgatcagtccaagaacggctacgctggttacattgatggtggtgcttctcaagaagagttcta caagttcatcaagcccatcttggagaagatggacggtactgaagagttgctggtcaagttgaacagagaggacttgctgagaaagcagaga accttcgacaacggttccattccacaccagattcacttgggtgagttgcacgctatcttgcgtagacaagaggacttctacccattcctgaagg acaacagagagaagatcgaaaagatcctgaccttcagaatcccctactacgttggtccattggctagaggtaactctagattcgcttggatga ccagaaagtccgaagagactatcaccccatggaacttcgaagaggttgttgacaagggtgcttccgctcaatccttcatcgagagaatgact aacttcgacaagaacctgccaaacgagaaggtcttgccaaagcactctttgctgtacgagtacttcaccgtctacaacgagctgactaaggt caagtacgttaccgagggtatgagaaagccagctttcttgtctggtgagcaaaagaaggctatcgtcgacttgctgttcaagaccaacagaa aggttaccgtcaagcagctgaaagaggactacttcaagaaaatcgagtgcttcgactccgtcgagatttccggtgttgaggatagattcaac gcctccttgggaacctaccacgacttgttgaagatcatcaaggacaaggatttcttggacaacgaggaaaacgaggacattttggaggacat cgtcttgaccttgactctgttcgaggacagagagatgattgaggaaagacttaagacttacgcccacctgttcgacgacaaggttatgaagca actgaagaggcgtagatacaccggttggggtagattgtctagaaagctgatcaacggtatcagagacaagcagtccggtaagactatcctg gactttttgaagtccgacggtttcgccaaccgtaacttcatgcaattgatccacgacgactccctgactttcaaagaggacattcagaaggctc aggtttccggtcaaggtgattccttgcatgagcacattgctaacttggctggttccccagctatcaagaagggtatcttgcagaccgttaaggtt gtcgacgagttggttaaggttatgggtagacacaagcccgagaacatcgttatcgaaatggctagagagaaccagactacccagaagggt caaaagaactccagagaaagaatgaagaggatcgaagagggtatcaaagagctgggttcccagattttgaaagagcacccagttgagaa cacccagctgcagaacgaaaagctgtacttgtactacttgcagaacggtagagacatgtacgtcgaccaagagctggacattaacagactg tctgactacgacgttgaccacatcgttccacagtcttttctgaaggacgactccatcgacaacaaggtcttgactagatccgataagaacagg ggtaagtccgacaacgttccttctgaagaggtcgtcaagaagatgaagaactactggcgtcagttgctgaacgccaagctgattactcagag gaagttcgacaacttgactaaggctgagagaggtggtttgtccgaattggataaggccggtttcatcaagagacagctggtcgagactaga cagatcacaaagcacgttgcccaaatcttggactccagaatgaacactaagtacgacgagaacgacaagttgatccgtgaggttaaggtca tcaccctgaagtccaagttggtgtccgatttcagaaaggacttccaattctacaaggtccgtgagatcaacaactaccaccatgctcacgacg cttacttgaacgctgttgttggtactgccctgatcaaaaagtaccctaagctggaatccgagttcgtctacggtgactacaaggtttacgacgtc agaaagatgatcgccaagtccgaacaagagatcggtaaggctactgccaagtactttttctactccaacatcatgaactttttcaagactgaga tcaccctggccaacggtgagatcagaaaaagaccactgatcgagactaacggtgaaaccggtgaaatcgtttgggacaagggtagagact tcgccaccgttagaaaggttttgtccatgccacaggtcaacatcgtcaaaaagactgaggttcagaccggtggtttctccaaagagtccatctt gcctaagagaaactccgacaagctgatcgccagaaagaaggattgggacccaaagaagtacggcggtttcgattctccaactgttgcctac tccgttttggttgttgctaaggtcgaaaagggcaagtctaagaagctgaagtccgtcaaagaactgctgggtatcactatcatggaaaggtcc agctttgagaagaacccaatcgactttttggaggccaagggttacaaagaggtgaagaaggacctgattatcaagctgccaaagtactccct gttcgagttggaaaacggtagaaagagaatgttggcttccgccggtgaattgcaaaagggtaacgaattggctctgccatccaagtacgtca actttctgtacttggcctctcactacgagaagttgaagggttctccagaggacaacgaacagaagcagttgttcgttgagcagcacaagcact acttggacgagatcattgagcagatttccgagttctccaagcgtgttattttggctgatgccaacctggataaggtcttgtccgcctacaacaag cacagagataagccaattagagagcaggctgagaacatcatccacttgttcactttgactaacctgggtgccccagctgcttttaagtacttcg acactaccatcgacagaaagagatacacctccaccaaagaggttttggacgctactttgatccaccagtccatcactggtctgtacgaaacc agaattgacttgtcccagcttggtggtgaccctaagaagaagagaaaggtctaa), and an amino acid sequence encoded is as shown in SEQ by the Cas9 gene ID NO.17 (MDKKYSIGLDIGTNSVGWAVITDEYKVPSKKFKVLGNTDRHSIKKNLIGALLFDSGETA EATRLKRTARRRYTRRKNRICYLQEIFSNEMAKVDDSFFHRLEESFLVEEDKKHERHPIFG NIVDEVAYHEKYPTIYHLRKKLVDSTDKADLRLIYLALAHMIKFRGHFLIEGDLNPDNSD VDKLFIQLVQTYNQLFEENPINASGVDAKAILSARLSKSRRLENLIAQLPGEKKNGLFGN LIALSLGLTPNFKSNFDLAEDAKLQLSKDTYDDDLDNLLAQIGDQYADLFLAAKNLSDAI LLSDILRVNTEITKAPLSASMIKRYDEHHQDLTLLKALVRQQLPEKYKEIFFDQSKNGYAG YIDGGASQEEFYKFIKPILEKMDGTEELLVKLNREDLLRKQRTFDNGSIPHQIHLGELHAI LRRQEDFYPFLKDNREKIEKILTFRIPYYVGPLARGNSRFAWMTRKSEETITPWNFEEVV DKGASAQSFIERMTNFDKNLPNEK VLPKHSLLYEYFTVYNELTKVKYVTEGMRKPAFLS GEQKKAIVDLLFKTNRKVTVKQLKEDYFKKIECFDSVEISGVEDRFNASLGTYHDLLKII KDKDFLDNEENEDILEDIVLTLTLFEDREMIEERLKTYAHLFDDKVMKQLKRRRYTGWG RLSRKLINGIRDKQSGKTILDFLKSDGFANRNFMQLIHDDSLTFKEDIQKAQVSGQGDSL HEHIANLAGSPAIKKGILQTVKVVDELVKVMGRHKPENIVIEMARENQTTQKGQKNSRE RMKRIEEGIKELGSQILKEHPVENTQLQNEKLYLYYLQNGRDMYVDQELDINRLSDYDV DHIVPQSFLKDDSIDNKVLTRSDKNRGKSDNVPSEEVVKKMKNYWRQLLNAKLITQRK FDNLTKAERGGLSELDKAGFIKRQLVETRQITKHVAQILDSRMNTKYDENDKLIREVK VI TLKSKLVSDFRKDFQFYKVREINNYHHAHDAYLNAVVGTALIKKYPKLESEFVYGDYKV YDVRKMIAKSEQEIGKATAKYFFYSNIMNFFKTEITLANGEIRKRPLIETNGETGEIVWDK GRDFATVRKVLSMPQVNIVKKTEVQTGGFSKESILPKRNSDKLIARKKDWDPKKYGGFD SPTVAYSVLVVAKVEKGKSKKLKSVKELLGITIMERSSFEKNPIDFLEAKGYKEVKKDLII KLPKYSLFELENGRKRMLASAGELQKGNELALPSKYVNFLYLASHYEKLKGSPEDNEQ KQLFVEQHKHYLDEIIEQISEFSKRVILADANLDKVLSAYNKHRDKPIREQAENIIHLFTLT NLGAPAAFKYFDTTIDRKRYTSTKEVLDATLIHQSITGLYETRIDLSQLGGDPKKKRKV).

In some embodiments, the recombinant expression cassette of Cas9 and gRNA is located on a nucleic acid construct and screened by using a Blasticidin resistance marker.

In some embodiments, the donor DNA may obtain left and right homologous arm fragments of the CCW14 gene, the EMW1 gene, and the PUN1 gene by utilizing an overlapping extension PCR technology based on Komagataella phaffii CBS7435 genomic DNA as a template. The homologous arm fragments refer to DNA sequences that are completely or highly homologous to specific regions of target genes (such as CCW14 gene, EMW1 gene, PUN1 gene, etc.).

The competent cell refers to a host cell treated by chemical method or electroporation. The cell membrane permeability of the competent cell is temporarily enhanced, enabling them to efficiently uptake exogenous DNA.

In some embodiments, one or more genes selected from the EXG1 gene and the SPR1 gene are overexpressed and integrated into a strain of the recombinant engineering bacterium, enabling the recombinant engineering bacterium to overexpress one or more genes of EXG1 gene and SPR1 gene.

In some embodiments, the recombinant expression cassette encoding one or more genes of the EXG1 gene and the SPR1 gene is located on a nucleic acid construct, or a nucleic acid construct with a plurality of selective markers.

The nucleic acid construct may be artificially assembled DNA or RNA molecules. In some embodiments, the nucleic acid construct may include a recombinant promoter, a coding sequence, a recombinant terminator, and a selective marker.

The selective marker may be functional genes carried in the nucleic acid construct. The expression product of the selective marker may make the host cells survive under a certain condition (such as antibiotic), so as to screen out specific cells.

In some embodiments, the nucleic acid construct includes any one or more of pHIL-D2, pPIC3.5, pHIL-S1, pPIC9, pPink-LC, pPink-HC, pGAPZA and pGAPZaA plasmids. The recombinant promoter may be a constitutive promoter, including any one or more of a GAP promoter, a TEF1 promoter, a PGK1 promoter, and an HGT1 promoter.

In some embodiments, an amino acid sequence encoded by the SPR1 gene is as shown in SEQ ID NO.8 (MKVSLFIWSSLIVGLVSGLLPVEYPQFKKFSNRTIIQNNNSPGRNQSASRFHKPSKLYGVA LGGWLVLEPYITPSLFNDTVEETVDEYTLCYKLGKKKVTELLTNHWSTFITESDIVKIKN VGLNSVRIPIGYWAYDLLEDDPYIQGQDEFLAQCIHWCAKHGLSVWIDLHGAPNSQNGF DNSGRRGRAGWQDDQRYIDKTLDVLGTIAKRHGNKPNVIGIEILNEPFGPVLNIDKLKQF YVKGIEVIRNTGYSKDVVISDAFQGIFHWDNFLPSASNIILDRHHYEVFSDGQLRSSFEGH LRGIEAFGRAIAIEKPTVVVGEWSAAITDCAPWVNGAGRPSRYHGMVLEDGTIGNCSGA TNIAQWAEKRRDELSKILQTSLKAYNAADGWFFWCWKTDSALEWDMEKLLEHGLFPL VGA). A nucleotide sequence of the SPR1 gene is a nucleotide sequence encoding a protein with a sequence shown in SEQ ID NO.8, including sequences that are publicly available in a database at present and nucleotide sequences that are optimized according to actual needs. In some embodiments, the nucleotide sequence of the SPR1 gene is as shown in SEQ ID NO.7 (atgaaggtgtccctgtttatctggtcctccttgatcgttggtttggtttccggtttgttgccagttgagtacccacagttcaagaagttctccaaca gaaccatcatccagaacaacaactccccaggtagaaaccagtctgcttccagatttcacaagccatccaagttgtacggtgttgctcttggtg gttggttggttttggagccttacatcactccatccttgttcaacgacaccgtcgaagaaactgttgacgagtacaccttgtgctacaagctgggt aagaagaaggtcaccgagttgttgactaaccactggtccactttcatcaccgagtccgacatcgtcaagatcaagaacgtcggtttgaactcc gtcagaatcccaattggttactgggcctacgatttgttggaggacgacccttacattcaaggtcaggatgagttcttggcccagtgtattcactg gtgtgctaagcacggtttgtccgtttggattgacttgcatggtgctccaaactctcagaacggtttcgacaactctggtagaagaggtagagct ggttggcaagacgaccagagatatatcgacaagaccttggacgtcttgggtactatcgctaagagacacggtaacaagccaaacgtcatcg gtatcgagattctgaacgaaccattcggtccagtcctgaacatcgacaagttgaagcagttctacgtcaagggtatcgaggtcatcagaaac accggttactccaaggacgttgttatctccgatgctttccagggtattttccactgggacaactttttgccatccgcctccaacatcatcttggac agacatcactacgaggtgttctccgatggtcagttgagatcctcattcgagggtcacttgagaggtattgaggctttcggtagagctatcgcta tcgagaagccaaccgttgttgttggtgaatggtccgctgctattactgattgtgctccatgggttaacggtgctggtagaccatctagatacca cggtatggtcttggaggatggaaccattggtaactgttctggtgctaccaacattgctcaatgggccgagaagagaagagatgagttgtctaa gatcctgcagacctccttgaaggcttacaacgctgctgatggttggttcttctggtgttggaaaactgactctgctttggagtgggacatggaa aagttgttggaacacggtctgttcccattggttggtgcttaa).

In some embodiments, an amino acid sequence encoded by the EXG1 gene is as shown in SEQ ID NO.10 (MGMNLYLITLLFASLCSALTLPKRDIIWDYSDEKIKGVNLGGWLVLEPFITPSLFEAFGD DVPVDEYRYTERLGKSLALDRLQQHWSTWYEEKDFQDIASYGLNFVRIPIGYWAFQLLD DDPYVQGQEEYLDKALEWSRKHGLKVWIDLHGAPGSQNGFDNSGKRDSWDFQKGDN VQVTLDVLKYISKKYGTADYYDVVIGIQLLNEPLGPILNMDNLRKFYADGYDLVRDVGN NFVVIHDAFYQEPEYWGDDFTSAEGYWNVVLDHHHYQVFDADQLQRSIDEHVEVACN WSRDANKEYHWNLCGEWSAALTDCTPWLNGVGKGTRYEGQLDGSPWIGSCENSQDPS KLSSERICEYRRYVEAQLDAFLYGKSAGFIFWCFKTEASLEWDFKRLVNAGIMPQPLDD RQYPNQCGF). A nucleotide sequence of the EXG1 gene is a nucleotide sequence encoding a protein with a sequence shown in SEQ ID NO.10, including sequences that are publicly available in a database at present and nucleotide sequences that are optimized according to actual needs. In some embodiments, the nucleotide sequence of the EXG1 gene is as shown in SEQ ID NO.9 (atgggcatgaacttgtacctgattaccttgctgttcgcctccttgtgttccgctttgactttgccaaagagagacatcatctgggactactccga cgagaagatcaagggtgttaaccttggtggttggttggtcttggagccattcattactccatccttgttcgaggctttcggtgacgatgttccagt tgacgagtacagatacaccgagagattgggaaagtccttggccttggacagattgcaacaacactggtctacttggtacgaagagaaggac ttccaggacattgcttcctacggtttgaacttcgtcagaatcccaatcggttactgggccttccagttgttggatgatgacccatacgttcagggt caagaagagtacttggacaaggctttggagtggtctagaaagcacggattgaaggtctggattgacttgcatggtgctccaggttctcaaaa cggtttcgacaactctggtaagagagattcctgggacttccaaaagggtgacaacgttcaggttaccttggacgtcttgaagtacatctccaa gaagtacggtactgccgactactacgacgttgttatcggtatccaactgctgaacgaaccattgggtccaatcctgaacatggacaacctga gaaagttctacgctgacggttacgacttggtcagagatgtcggtaacaacttcgttgtcatccacgacgctttctaccaagaaccagaatactg gggtgacgacttcacttctgctgaaggttactggaacgtcgttttggatcaccaccactaccaagttttcgacgctgaccaattgcagagatcc attgacgaacacgttgaggttgcttgcaactggtctagagatgccaacaaagagtaccactggaacttgtgtggtgaatggtctgctgctttga ctgactgtactccatggttgaacggtgtcggtaagggtactagatacgagggtcaattggatggttccccatggattggttcttgcgagaactc tcaagacccatccaagttgtcctccgagagaatctgtgagtacagaagatacgttgaggctcagttggacgccttcttgtacggtaaatctgc cggtttcatcttctggtgcttcaagactgaagcttccttggagtgggacttcaagagattggttaacgccggtattatgccacagccattggac gatagacagtacccaaaccagtgtggtttctaa).

In some embodiments, the recombinant engineering bacterium includes at least one copy of a nucleotide sequence encoding the recombinant human albumin. In some embodiments, the copy number of the nucleotide sequence encoding the recombinant human albumin in the recombinant engineering bacterium is 1-5. In some embodiments of the present disclosure, the copy number of the nucleotide sequence encoding the recombinant human albumin in the recombinant engineering bacterium is 3.

In some embodiments, the recombinant engineering bacterium also includes a drug resistance gene fragment, facilitating screening of recombinant engineering strains. In some embodiments, the recombinant engineering bacterium includes one or more of a Blasticidin resistance gene and a geneticin G418 resistance gene.

In some embodiments, a nucleotide sequence of the Blasticidin resistance gene included in the recombinant engineering bacterium is as shown in SEQ ID NO.20 (atggccaagcctttgtctcaagaagaatccaccctcattgaaagagcaacggctacaatcaacagcatccccatctctgaagactacagcg tcgccagcgcagctctctctagcgacggccgcatcttcactggtgtcaatgtatatcattttactgggggaccttgtgcagaactcgtggtgct gggcactgctgctgctgcggcagctggcaacctgacttgtatcgtcgcgatcggaaatgagaacaggggcatcttgagcccctgcggacg gtgccgacaggtgcttctcgatctgcatcctgggatcaaagccatagtgaaggacagtgatggacagccgacggcagttgggattcgtgaa ttgctgccctctggttatgtgtgggagggctaa), or as shown in a nucleotide sequence at least having 98% identity to SEQ ID NO.20, and a nucleotide sequence of the geneticin G418 resistance gene is as shown in SEQ ID NO.21 (atgggtaaggaaaagactcacgtttcgaggccgcgattaaattccaacatggatgctgatttatatgggtataaatgggctcgcgataatgtc gggcaatcaggtgcgacaatctatcgattgtatgggaagcccgatgcgccagagttgtttctgaaacatggcaaaggtagcgttgccaatga tgttacagatgagatggtcagactaaactggctgacggaatttatgcctcttccgaccatcaagcattttatccgtactcctgatgatgcatggtt actcaccactgcgatccccggcaaaacagcattccaggtattagaagaatatcctgattcaggtgaaaatattgttgatgcgctggcagtgttc ctgcgccggttgcattcgattcctgtttgtaattgtccttttaacagcgatcgcgtatttcgtctggctcaggcgcaatcacgaatgaataacggt ttggttgatgcgagtgattttgatgacgagcgtaatggctggcctgttgaacaagtctggaaagaaatgcataagcttttgccattctcaccgg attcagtcgtcactcatggtgatttctcacttgataaccttatttttgacgaggggaaattaataggttgtattgatgttggacgagtcggaatcgc agaccgataccaggatcttgccatcctatggaactgcctcggtgagttttctccttcattacagaaacggctttttcaaaaatatggtattgataat cctgatatgaataaattgcagtttcatttgatgctcgatgagtttttctaa), or as shown in a nucleotide sequence at least having 98% identity to SEQ ID NO.21.

In some embodiments, the recombinant engineering bacterium also includes a signal peptide sequence that is capable of promoting protein expression. The signal peptide sequence is used for secretion expression of an exogenous protein of the recombinant engineering bacterium.

In some embodiments, the signal peptide is a α-mating factor signal peptide from Saccharomyces cerevisiae. In some embodiments, a nucleotide sequence encoding the α-mating factor signal peptide from Saccharomyces cerevisiae is as shown in SEQ ID NO.18 (atgagattcccctccatcttcaccgctgttttgttcgctgcttcttctgctttggctgctccagttaacactactactgaggacgagactgctcag attccagctgaagctgttattggttactccgacttggaaggtgacttcgacgttgctgttttgccattctccaactccaccaacaacggtctgttgt tcatcaacaccactatcgcttccattgccgctaaagaagaaggcgtttccttggagaagagagaggctgaagct).

In some embodiments, the recombinant engineering bacterium may be selected from one or more of Komagataella phaffii, Hansenula polymorpha, Candida parapsilosis, and Saccharomyces cerevisiae. In some embodiments, the Komagataella phaffii is Komagataella phaffii CBS7435.

### Example 1: Selection of Target Genes

In some embodiments, candidate protein genes related to structural components of a cell wall were selected and corresponding gene nucleotide sequences of the candidate protein genes and protein sequences encoded by the candidate protein genes were obtained by retrieving currently known protein databases (such as Uniprot, The Human Protein Atlas, PhosphoSitePlus) and publicly available documents.

In some embodiments, artificial nucleotide sequences were formed by optimizing the gene nucleotide sequences of the candidate protein genes, and the formed artificial nucleotide sequences were used for subsequent experiments.

In some embodiments, the candidate protein genes may include genes directly constituting the components of the cell wall such as CCW14 gene, EMW1 gene, and PUN1 gene and genes related to β-glucan hydrolase such as SPR1 gene and EXG1 gene. The detailed information of each gene was shown in Table 1.

TABLE 1

| Species | Genes | Coding proteins | Protein annotation information |
|---|---|---|---|
| Komagataella | CCW14 | Cell wall glycoprotein connexin | www.uniprot.org/uniprotkb/ |
| phaffii | | | A0A1G4KPI2/entry |
| Komagataella | EMW1 | Cell wall integrity protein | www.uniprot.org/uniprotkb/ |
| phaffii | | | A0A1G4KPG0/entry |
| Komagataella | PUN1 | Cell wall integrity protein | www.uniprot.org/uniprotkb/ |
| phaffii | | | F2QLI3/entry |
| Komagataella | EXG1 | Glucan-1,3-β-glucosidase | www.uniprot.org/uniprotkb/ |
| phaffii | | | A0A1B2JCC4/entry |
| Komagataella | SPR1 | Glucan-1,3-β-glucosidase | www.uniprot.org/uniprotkb/ |
| phaffii | | | A0A1B2JHM0/entry |

### Example 2: Construction of Komagataella phaffii CBS7435 Strain Expressing Recombinant Human Albumin

In some embodiments, a recombinant engineering bacterium for human albumin expression may include Hansenula, Pichia, Candida, Saccharomyces, etc. Hansenula, Pichia, and Candida may be used for preparing a recombinant engineering bacterium of a recombinant human albumin. Saccharomyces cerevisiae may be used for preparing a recombinant engineering bacterium of a recombinant albumin. In the present disclosure, Komagataella phaffii CBS7435 was used for experiments.

In some embodiments, a biological company was entrusted to directly synthesize a nucleic acid molecule (SEQ ID NO.18) encoding a α-mating factor signal peptide from Saccharomyces cerevisiae and a nucleic acid molecule (with a nucleotide sequence as shown in SEQ ID NO.12, and an amino acid sequence encoded by the nucleotide sequence being as shown in SEQ ID NO.11) encoding a recombinant human albumin.

In some embodiments, the nucleic acid molecule encoding the α-mating factor signal peptide from Saccharomyces cerevisiae and the nucleic acid molecule encoding the recombinant human albumin were linked to a plasmid pPICZA digested by EcoRI and NotI to form an expression vector named pPICZA-rHA (as shown in FIG. 1).

In some embodiments, the expression vector was linearized using DNA restriction endonuclease BglII, etc., Komagataella phaffii CBS7435 was transformed by electric shock or other methods, the transformed Komagataella phaffii CBS7435 were coated onto a culture medium (e.g., a selective plate YPDZ containing different high concentrations of zeocin antibiotics). The culture medium was cultured at a preset temperature (e.g., 30° C.) until colonies appeared, and positive transformants were identified by colony polymerase chain reaction (PCR) and then stored in a glycerinum tube.

In some embodiments, the genomic DNA of 4 randomly selected transformants was extracted with a yeast genomic extraction kit, and a real-time fluorescent quantitative PCR was carried out by respectively utilizing vectors containing endogenous gene HIS4 and exogenous gene recombinant human albumins present in a form of single copy by using a double standard curve method. A standard curve was established based on results of the real-time fluorescent quantitative PCR, and the copy number of the recombinant human albumin in the genomic DNA of the transformants was detected.

In some embodiments, a double standard curve was established by respectively using a pPIC9K vector containing a single copy of HIS4 gene and a pPICZA-rHA vector containing a single copy of recombinant human albumin gene as standards. The copy number of a starting plasmid was 10⁹, multiple dilution gradients were obtained by dilution according to a preset rule (e.g., 10-fold dilution, a total of 6 dilution gradients), and 2 parallel samples for each dilution gradient were used as an amplified standard curve. Each copy of extracted transformant genome was diluted to 10 ng/μL as a PCR template.

The fluorescent quantitative PCR reaction system was shown in Table 2, and specific nucleotide sequences of upstream primers and downstream primers were shown in Table 3.

TABLE 2

| | |
|---|---|
| FastSYBR Mixture | 10 μL |
| Upstream primer | 0.4 μL |
| Downstream primer | 0.4 μL |
| DNA template | 5 μL |
| RNase-Free ddH₂O | 4.2 μL |

TABLE 3

| | |
|---|---|
| HIS4 upstream primer | GGTTGAACAAACAGGTGTTG |
| | (SEQ ID NO. 22) |
| HIS4 downstream primer | GTTCCTTGGTGTATCCTGGC |
| | (SEQ ID NO. 23) |
| rHA upstream primer | GTACGAAATCGCCAGACG |
| | (SEQ ID NO. 24) |
| rHA downstream primer | CCTTTCGCCGAACTTCTG |
| | (SEQ ID NO. 25) |

Fluorescence quantitative PCR reaction parameters include as follows:
Stage 1: holding for 3 min at 95° C.; stage 2: holding for 5 s at 95° C. and holding for 20 s at 60° C. as a cycle, repeating for 40 cycles; stage 3: holding for 10 s at 95° C., holding for 5 min at 72° C., and holding for 1 s at 97° C.

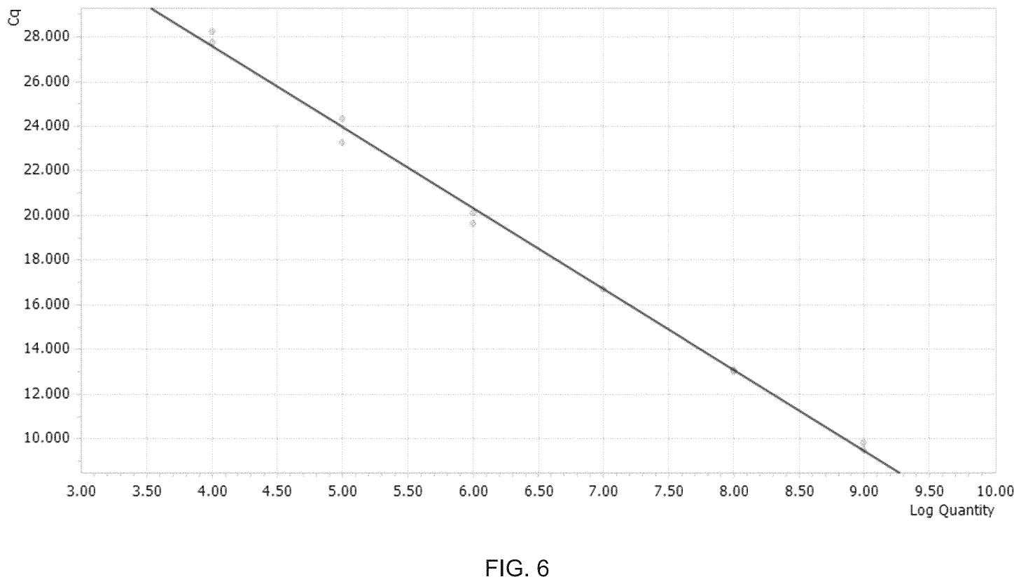

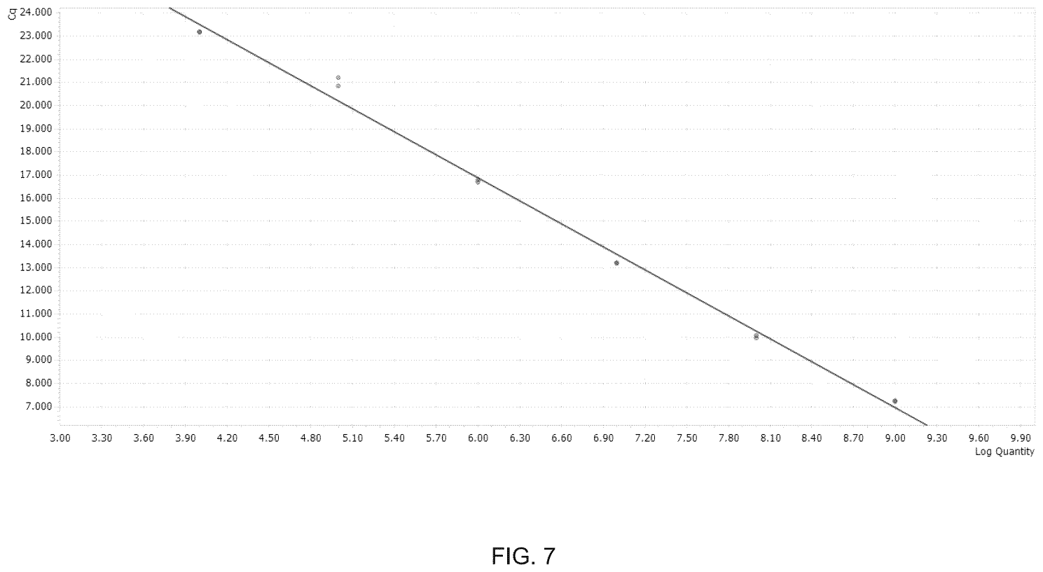

FIG. 6 is a result graph of a standard curve of an HIS4 gene in example 1. FIG. 7 is a result graph of a standard curve of an rHA gene in example 1.

In the embodiments, the cycle number Ct of each copy of transformant test solution was substituted into a standard curve (the standard curve of the HIS4 gene as shown in FIG. 6, and a standard curve of the rHA gene as shown in FIG. 7) to obtain copy number concentrations of starting templates corresponding to endogenous gene HIS4 and exogenous gene rHA in a genome. The copy number result of the genomic DNA of 4 transformants obtained by screening after transformation was calculated by a ratio of a copy number of starting templates corresponding to the exogenous gene rHA to a copy number of starting templates corresponding to the endogenous gene HIS4. Specific information was shown in Table 4.

TABLE 4

| | Copy | | | | Over- | Over- |
|---|---|---|---|---|---|---|
| | number of | Knock | Knock | Knock | ex- | ex- |
| | recombinant | out | out | out | press | press |
| | human | CCW14 | EMW1 | PUN1 | SPR1 | EXG1 |
| Strain | albumin | gene | gene | gene | gene | gene |
| Recombinant | 1 | No | No | No | No | No |
| engineering | | | | | | |
| bacterium 1 | | | | | | |
| Recombinant | 3 | No | No | No | No | No |
| engineering | | | | | | |
| bacterium 2 | | | | | | |
| Recombinant | 3 | No | No | No | No | No |
| engineering | | | | | | |
| bacterium 3 | | | | | | |
| Recombinant | 2 | No | No | No | No | No |
| engineering | | | | | | |
| bacterium 4 | | | | | | |

### Example 3: Construction of Cas9 and Grna Co-Expression Vector (Also Known as Cas9 and Grna Co-Expression Plasmid)

In some embodiments, to establish a proper genome editing CRISPR system, it was attempted to construct a Cas9 and gRNA co-expression vector using double promoters pHTX1 (with a nucleotide acid sequence as shown in SEQ ID NO.19 (gttgtagttttaatatagtttgagtatgagatggaactcagaacgaaggaattatcaccagtttatatattctgaggaaagggtgtgtcctaaatt ggacagtcacgatggcaataaacgctcagccaatcagaatgcaggagccataaattgttgtattattgctgcaagatttatgtgggttcacatt ccactgaatggttttcactgtagaattggtgtcctagttgttatgtttcgagatgttttcaagaaaaactaaaatgcacaaactgaccaataatgtg ccgtcgcgcttggtacaaacgtcaggattgccaccacttttttcgcactctggtacaaaagttcgcacttcccactcgtatgtaacgaaaaaca gagcagtctatccagaacgagacaaattagcgcgtactgtcccattccataaggtatcataggaaacgagagtcctccccccatcacgtatat ataaacacactgatatcccacatccgcttgtcaccaaactaatacatccagttcaagttacctaaacaaatcaaa)).

In some embodiments, a biological company was entrusted to directly synthesize a recombinant expression cassette encoding a Cas9 gene and a CCW14-gRNA gene.

In some embodiments, the recombinant expression cassette encoding the Cas9 gene and the CCW14-gRNA gene is linked to a plasmid pPICZA digested by BglII and BamHI to form an expression vector named pART201. A zeocin resistance gene in pART201 was placed with a Blasticidin resistance gene (with a nucleotide sequence as shown in SEQ ID NO.20) utilizing a gene homologous recombination technology to form an expression vector named pART202 (as shown in FIG. 2).

In some embodiments, a biological company was entrusted to directly synthesize a recombinant expression cassette encoding a Cas9 gene and an EMW1-gRNA gene.

In some embodiments, the recombinant expression cassette encoding the Cas9 gene and the EMW1-gRNA gene is linked to a plasmid pPICZA digested by BglII and BamHI to form an expression vector named pART203. A zeocin resistance gene in pART203 was placed with a Blasticidin resistance gene (with a nucleotide sequence as shown in SEQ ID NO.20) utilizing a gene homologous recombination technology to form an expression vector named pART204 (as shown in FIG. 3).

In some embodiments, a biological company was entrusted to directly synthesize a recombinant expression cassette encoding a Cas9 gene and a PUN1-gRNA gene.

In some embodiments, the recombinant expression cassette encoding the Cas9 gene and the PUN1-gRNA gene is linked to a plasmid pPICZA digested by BglII and BamHI to form an expression vector named pART205. A zeocin resistance gene in pART205 was placed with a Blasticidin resistance gene (with a nucleotide sequence as shown in SEQ ID NO.20) utilizing a gene homologous recombination technology to form an expression vector named pART206 (as shown in FIG. 4).

### Example 4: Construction of Strains with Targeted Knockout of One or More Genes of CCW14 Gene, EMW1 Gene, and PUN1 Gene

In some embodiments, to prepare donor DNA with knockout of the CCW14 gene, the following operations were performed: left homologous arm fragments and right homologous arm fragments of the CCW14 gene were obtained by a PCR technology with Komagataella phaffii CBS7435 genomic DNA as a template and CCW14-UP-F/CCW14-UP-R and CCW14-DN-F/CCW14-DN-R as primers. The donor DNA with knockout of the CCW14 gene was obtained by utilizing an overlapping extension PCR technology with CCW14-UP-F and CCW14-DN-R as primers and a mixture of the left homologous arm fragments and the right homologous arm fragments as a DNA template.

In some embodiments, to prepare donor DNA with knockout the EMW1 gene, the following operations were performed: left homologous arm fragments and right homologous arm fragments of the EMW1 gene were obtained by the PCR technology with Komagataella phaffii CBS7435 genomic DNA as a template and EMW1-UP-F/EMW1-UP-R and EMW1-DN-F/EMW1-DN-R as primers. The donor DNA with knockout of the EMW1 gene was obtained by utilizing the overlapping extension PCR technology with EMW1-UP-F and EMW1-DN-R as primers and a mixture of the left homologous arm fragments and the right homologous arm fragments as a DNA template.

In some embodiments, to prepare donor DNA with knockout of PUN1 gene, the following operations were performed: left homologous arm fragments and right homologous arm fragments of the PUN1 gene were obtained by PCR with Komagataella phaffii CBS7435 genomic DNA as a template and PUN1-UP-F/PUN1-UP-R and PUN1-DN-F/PUN1-DN-R as primers. The donor DNA with knockout of the PUN1 gene was obtained by utilizing the overlapping extension PCR technology with PUN1-UP-F and PUN1-DN-R as primers and a mixture of the left homologous arm fragments and the right homologous arm fragments as a DNA template.

The PCR reaction system was shown in Table 5, and specific nucleotide sequences of upstream primers and downstream primers were shown in Table 6.

TABLE 5

| | |
|---|---|
| PrimeSTAR HS | 12.5 μl |
| Upstream primer | 1 μl |
| Downstream primer | 1 μl |
| DNA template | 5 μl |
| ddH₂O | 5.5 μl |

TABLE 6

| | |
|---|---|
| CCW14-UP-F | GCCGTCTGTTAGGATTGC |
| | (SEQ ID NO. 26) |
| CCW14-UP-R | CGTACATAATGTTCTTTCAGTATTAAACCTAG |
| | (SEQ ID NO. 27) |
| CCW14-DN-F | CTGAAAGAACATTATGTACG |
| | (SEQ ID NO. 28) |
| CCW14-DN-R | CCAATCCAATGAGACCTC |
| | (SEQ ID NO. 29) |
| EMW1-UP-F | CAAATGTTTAACCGTTGAC |
| | (SEQ ID NO. 30) |
| EMW1-UP-R | GTGTAGACTATATTTTGATGGTTGATTTGGCCG |
| | (SEQ ID NO. 31) |
| EMW1-DN-F | CAACCATCAAAATATAGTCTACACATC |
| | (SEQ ID NO. 32) |
| EMW1-DN-R | GCGACTATTCATTTGCAG |
| | (SEQ ID NO. 33) |
| PUN1-UP-F | CCAGTTGGTCAAGGCATC |
| | (SEQ ID NO. 34) |
| PUN1-UP-R | CTATGATATTCCCTTGATTAATATGAGACGT |
| | (SEQ ID NO. 35) |
| PUN1-DN-F | AATCAAGGGAATATCATAGAAAC |
| | (SEQ ID NO. 36) |
| PUN1-DN-R | CACCATAAGAGGCCAATC |
| | (SEQ ID NO. 37) |

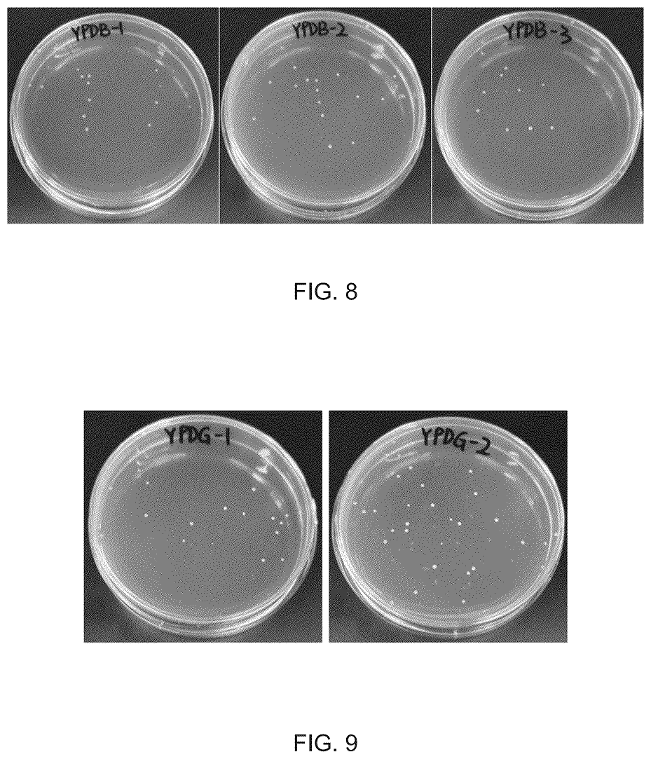

FIG. 8 is a colony atlas in example 4.

In some embodiments, 2 μg of the Cas9 and gRNA co-expression vector and 2 μg of the donor DNA were simultaneously transformed into the recombinant engineering bacterium 2 in example 1, and then the above transformed recombinant engineering bacterium 2 was coated onto a culture medium (e.g., a selective plate YPDB containing Blasticidin antibiotic). The culture medium was cultured at 30° C. until colonies appeared, as shown in FIG. 8, positive transformants were identified by colony PCR as a recombinant engineering bacterium 5, a recombinant engineering bacterium 6, and a recombinant engineering bacterium 7, and specific information was shown in Table 7.

TABLE 7

| | Copy | | | | Over- | Over- |
|---|---|---|---|---|---|---|
| | number of | Knock | Knock | Knock | ex- | ex- |
| | recombinant | out | out | out | press | press |
| | human | CCW14 | EMW1 | PUN1 | SPR1 | EXG1 |
| Strain | albumin | gene | gene | gene | gene | gene |
| Recombinant | 3 | Yes | No | No | No | No |
| engineering | | | | | | |
| bacterium 5 | | | | | | |
| Recombinant | 3 | Yes | Yes | No | No | No |
| engineering | | | | | | |
| bacterium 6 | | | | | | |
| Recombinant | 3 | Yes | Yes | Yes | No | No |
| engineering | | | | | | |
| bacterium 7 | | | | | | |

### Example 5: Construction of Co-Expression Strain of SPR1 Gene and EXG1 Gene

In some embodiments, a biological company was entrusted to directly synthesize a recombinant expression cassette encoding an SPR1 gene (with a nucleotide sequence as shown in SEQ ID NO.7, an amino acid sequence encoded by the nucleotide sequence being as shown in SEQ ID NO.8).

In some embodiments, the recombinant expression cassette encoding the SPR1 gene was linked to a plasmid pGAPZA to form an expression vector named pART207. A zeocin resistance gene in pART207 was placed with a geneticin G418 resistance gene (with a nucleotide sequence as shown in SEQ ID NO.21) utilizing the gene homologous recombination technology to form an expression vector named pART208.

In some embodiments, a biological company was entrusted to directly synthesize a recombinant expression cassette encoding an SPR1 gene (with a nucleotide sequence as shown in SEQ ID NO.7, an amino acid sequence encoded by the nucleotide sequence being as shown in SEQ ID NO.8) and an EXG1 gene (with a nucleotide sequence as shown in SEQ ID NO.9, an amino acid sequence encoded by the nucleotide sequence being as shown in SEQ ID NO.10).

In some embodiments, the recombinant expression cassette encoding the SPR1 gene and the EXG1 gene was respectively linked to a plasmid pGAPZA to form an expression vector named PART209. A zeocin resistance gene in pART209 was placed with a geneticin G418 resistance gene (with a nucleotide sequence as shown in SEQ ID NO.21) to form an expression vector named PART210 (as shown in FIG. 5).

FIG. 9 is a colony atlas in example 5.

In some embodiments, the expression vectors pART208 and pART210 were respectively linearized using DNA restriction endonucleases BglII, etc., the recombinant engineering bacterium 7 was transformed by electric shock or other methods, and the transformed recombinant engineering bacterium 7 was coated onto a culture medium (e.g., a selective plate YPDG containing geneticin G418 antibiotic). The culture medium was cultured at 30° C. until colonies appeared, as shown in FIG. 9, positive transformants were identified by colony PCR as a recombinant engineering bacterium 8 and a recombinant engineering bacterium 9, and specific results were shown in Table 8.

TABLE 8

| | Copy | | | | Over- | Over- |
|---|---|---|---|---|---|---|
| | number of | Knock | Knock | Knock | ex- | ex- |
| | recombinant | out | out | out | press | press |
| | human | CCW14 | EMW1 | PUN1 | SPR1 | EXG1 |
| Strains | albumin | gene | gene | gene | gene | gene |
| Recombinant | 3 | Yes | Yes | Yes | Yes | No |
| engineering | | | | | | |
| bacterium 8 | | | | | | |
| Recombinant | 3 | Yes | Yes | Yes | Yes | Yes |
| engineering | | | | | | |
| bacterium 9 | | | | | | |

### Example 6: Expression Level of Recombinant Human Albumin in Recombinant Engineering Bacteria

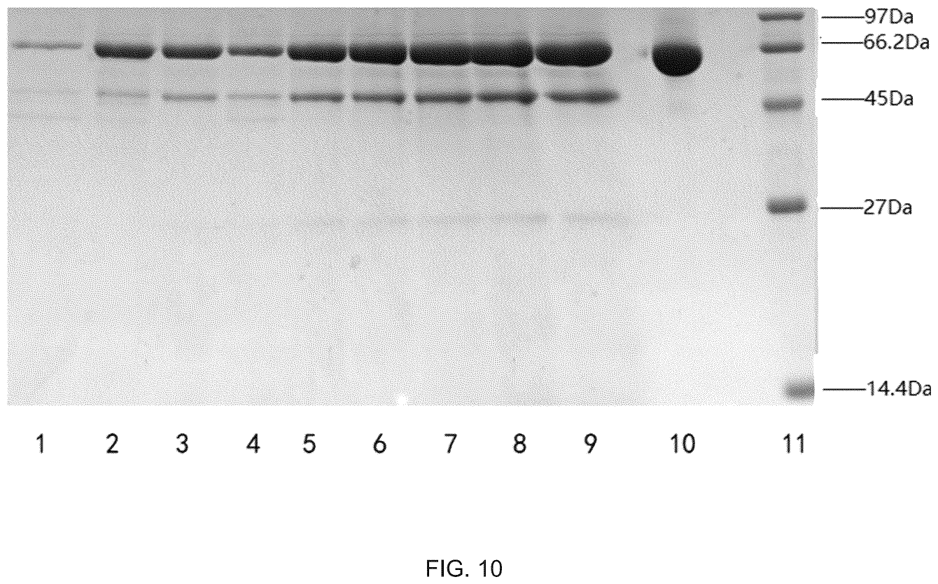

FIG. 10 is a sodium dodecyl sulfate-polyacrylamide gel electrophoresis (SDS-PAGE) electropherogram in example 6. In the figure, electropherograms numbered 1-9 are electropherograms showing the expression of recombinant human albumins in recombinant engineering bacteria 1-9, the electropherogram numbered 10 is an electropherogram of a human serum albumin, and the electropherogram numbered 11 is a protein Marker.

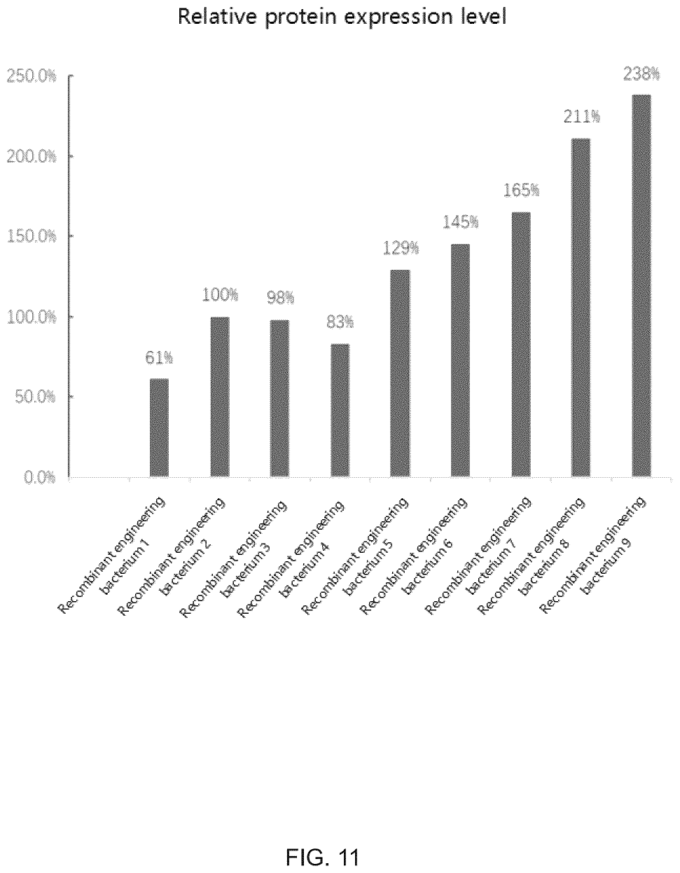

FIG. 11 is a result diagram showing that recombinant engineering bacteria express the recombinant human albumin in example 6.

In some embodiments, recombinant engineering bacteria 1-9 were respectively taken and inoculated into a culture solution (e.g., a BMGY culture solution, etc.) with a preset volume (e.g., 40 mL), the culture solution was cultured for overnight at 30° C. at a preset oscillation speed (e.g., 220 rpm). A bacterial solution of the culture solution after overnight culture was taken and centrifuged, a precipitate after centrifugation was resuspended into 40 mL of BMMY culture solution and then the BMMY culture solution was started to be induced at 25° C. under 220 rpm, 0.4% methanol was added into the BMMY culture solution every 24 h, the BMMY culture solution was centrifuged for 5 min under 10,000 rpm after 72 h, and then supernatant was collected. The supernatant was subjected to SDS-PAGE electrophoresis detection. The electrophoretogram is as shown in FIG. 10, and specific results are shown in Table 9 and FIG. 11.

TABLE 9

| | Copy | | | | | | |
|---|---|---|---|---|---|---|---|
| | number of | Knock | Knock | Knock | Over- | Over- | Relative |
| | recombinant | out | out | out | express | express | protein |
| | human | CCW14 | EMW1 | PUN1 | SPR1 | EXG1 | expression |
| Strain | albumin | gene | gene | gene | gene | gene | level |
| Recombinant | 1 | No | No | No | No | No | 61% |
| engineering | | | | | | | |
| bacterium 1 | | | | | | | |
| Recombinant | 3 | No | No | No | No | No | 100% |
| engineering | | | | | | | |
| bacterium 2 | | | | | | | |
| Recombinant | 3 | No | No | No | No | No | 98% |
| engineering | | | | | | | |
| bacterium 3 | | | | | | | |
| Recombinant | 2 | No | No | No | No | No | 83% |
| engineering | | | | | | | |
| bacterium 4 | | | | | | | |
| Recombinant | 3 | Yes | No | No | No | No | 129% |
| engineering | | | | | | | |
| bacterium 5 | | | | | | | |
| Recombinant | 3 | Yes | Yes | No | No | No | 145% |
| engineering | | | | | | | |
| bacterium 6 | | | | | | | |
| Recombinant | 3 | Yes | Yes | Yes | No | No | 165% |
| engineering | | | | | | | |
| bacterium 7 | | | | | | | |
| Recombinant | 3 | Yes | Yes | Yes | Yes | No | 211% |
| engineering | | | | | | | |
| bacterium 8 | | | | | | | |
| Recombinant | 3 | Yes | Yes | Yes | Yes | Yes | 238% |
| engineering | | | | | | | |
| bacterium 9 | | | | | | | |

It can be seen from FIG. 9 and FIG. 11 that when one or more of the CCW14 gene, the EMW1 gene, and the PUN1 gene are knocked out, or when one or two of the EXG1 gene and the SPR1 gene are over-expressed, the relative protein expression levels increase to varying degrees by using the expression level of the recombinant human albumin in the recombinant engineering bacterium 2 as control. The recombinant engineering bacterium 9 with knockout of the CCW14 gene, the NCW4 gene, and the PUN1 gene and overexpression of the EXG1 gene and the SPR1 gene exhibits an optimal effect, the relative protein expression level of which reaches 238%.

The recombinant engineering bacterium constructed in some embodiments of the present disclosure can significantly improve the yield of the recombinant human albumin, i.e., the yield of the recombinant human albumin can reach more than 200%. Different from traditional methods that can directly improve a copy number of a recombinant protein-coding gene, the recombinant engineering bacterium of the present disclosure conducts knockout on the genes related to the components of the cell wall (such as one or more of a CCW14 gene, an EMW1 gene and a PUN1 gene) by improving the structure of the cell wall, and over-expresses β-glucan hydrolase genes (such as one or more genes of SPR1 gene and EXG1 gene) to cause a structure defect of the cell wall, thereby significantly enhancing a heterologous protein secretion ability of the recombinant engineering bacterium. The recombinant engineering bacterium exhibits powerful potential in improving the yield of a protein with a high economic value and has wide application prospects.

The above descriptions are only embodiments of the present disclosure, but are not used for limiting the present disclosure. For those skilled in the art, various changes and variations can be made to the present disclosure. Any modifications, equivalent replacements, improvements and the like made within the spirit and principle of the present disclosure should be included within the scope of claims of the present disclosure.

1. A recombinant engineering bacterium for improving a yield of a recombinant human albumin, wherein the recombinant engineering bacterium comprises the following operations while expressing the recombinant human albumin:
conducting targeted knockout on one or more genes selected from the group consisting of a CCW14 gene, an EMW1 gene, and a PUN1 gene by utilizing a CRISPR-Cas9 gene editing technology; and
over-expressing and integrating one or more genes selected from the group consisting of an EXG1 gene and a SPR1 gene into a strain of the recombinant engineering bacterium, wherein
an amino acid sequence encoded by the SPR1 gene is as shown in SEQ ID NO:8,
an amino acid sequence encoded by the EXG1 gene is as shown in SEQ ID NO:10, and
the recombinant engineering bacterium comprises at least one copy of a nucleotide sequence encoding the recombinant human albumin, and the recombinant engineering bacterium is formed by modifying Komagataella phaffii.

2. The recombinant engineering bacterium according to claim 1, wherein
an amino acid sequence encoded by the CCW14 gene is as shown in SEQ ID NO:2;
an amino acid sequence encoded by the EMW1 gene is as shown in SEQ ID NO:4; and
an amino acid sequence encoded by the PUN1 gene is as shown in SEQ ID NO:6.

3. The recombinant engineering bacterium according to claim 1, wherein
a nucleotide sequence of the CCW14 gene is as shown in SEQ ID NO:1;
a nucleotide sequence of the EMW1 gene is as shown in SEQ ID NO:3; and
a nucleotide sequence of the PUN1 gene is as shown in SEQ ID NO:5.

4. The recombinant engineering bacterium according to claim 1, wherein the recombinant engineering bacterium further comprises a drug resistance gene fragment and/or a signal peptide sequence.

5. The recombinant engineering bacterium according to claim 1, wherein the conducting targeted knockout on one or more genes selected from the CCW14 gene, the EMW1 gene, and the PUN1 gene by utilizing the CRISPR-Cas9 gene editing technology comprises the following steps:
S1: constructing a Cas9 and gRNA co-expression plasmid;
S2: preparing donor DNA;
S3: preparing a competent cell; and
S4: constructing a strain with one or more genes selected from the CCW14 gene, the EMW1 gene, and the PUN1 gene are knocked out.

6. The recombinant engineering bacterium according to claim 1, wherein a recombinant expression cassette encoding one or more genes selected from the EXG1 gene and the SPR1 gene is located on a nucleic acid construct or a nucleic acid construct with a plurality of selective markers.

7. The recombinant engineering bacterium according to claim 6, wherein the recombinant engineering bacterium is formed by modifying Komagataella phaffii CBS7435.

8. A method for preparing a recombinant human albumin, comprising:
making the recombinant human albumin by fermenting the recombinant engineering bacterium according to claim 1.


SEQUENCE LISTING (21 sequences)

SEQ ID NO: 1 - DNA, 702 bp, Komagataella phaffii
  source 1..702: mol_type = genomic DNA
atgcagtttactttcgcttctacctctgttgttgtctcactgattgctgctctcgctaagcctgctgttgccactccaccggcttgcctgttggcctgtgctgccgaggttgttaaggaatcatccgactgtgatgccttgaacaacattcaatgtatctgtgagaatgaaggatcagccattcatgcctgcttggagtcaacctgtcccgatggattgtcgtccactgctttgcagtcttttgaagatgtttgcgagagtgtcggcactgaagctaaccttgatgagagctcttcatctcagtcctcatcatcgtcttcttcttcggagtcatcatcgtcttccgtgtcatcatcatcctcttccgcatcttcttcttccgagacttcctcttccgtgacttcctcttccgtgacttcctcttccactgcagtctcttccagcaccgaatcctcttcatccgttgagccatcgacttcccactcttcttcccactcttcgtctgaagtctcctcaactgtcgcaccaaccaccagtgttgcaccaactaccagttccatcactacttccagtacttctttgacatcagctactacttccagtgtgactatttctatcgagccaactagcgacgctgctgacaaggttatcatcccaggtttggctggtctagtcggtgctttggctgttggtctgatctag

SEQ ID NO: 2 - protein, 233 aa, Komagataella phaffii
MQFTFASTSVVVSLIAALAKPAVATPPACLLACAAEVVKESSDCDALNNIQCICENEGSAIHACLESTCPDGLSSTALQSFEDVCESVGTEANLDESSSSQSSSSSSSSESSSSSVSSSSSSASSSSETSSSVTSSSVTSSSTAVSSSTESSSSVEPSTSHSSSHSSSEVSSTVAPTTSVAPTTSSITTSSTSLTSATTSSVTISIEPTSDAADKVIIPGLAGLVGALAVGLI

SEQ ID NO: 3 - DNA, 2874 bp, Komagataella phaffii
  source 1..2874: mol_type = genomic DNA
atgtcagccctaaggtctattcatagctcgtttattaggctgaatatagaattgaatgtgggggttgaaggctttaacgagcaaactgaggccatctccttaataagcaaaggtcaatatcttaaagtcctgaatttgcctcttttcaaaaagatcttcactgataattccgaaatcaagcatataattgcaaagtatacccagcaatcatcggcatcggcactagtggttttggatttagtcaatcatatcattatgcaactatccaagaatctggagtcgttgagtccagaagtagtcaaaactactgtactgattttaggtgtgatctttttacaactatttctgcaagaaaacttcactggtccttcaataccttttgagaatgaaaaattaccgctggggatattaagagacttgctgaaattgaataaagaagcctctacgcatatattgtctgttggaggtgagacagcttatgaactcatggagtctcctattctactggttctttcagtgaaaatattagaagaagtgtcaggttctagtgtcagtgttctgtccaaagatttcattgatatggatagtgaaacgtttatttccaaatgtaatatcgttaccaatcccgatgttgtgcaatcctcgatacaatggtggaagtcaagagcttatatggtgtttgcatcggttctcactggagaagtatccctgttgaccttactgcccatcaaactgcttacaccaaacattgtttcaacattgatagatttacaaaataagaactcaccagaaaaccaggagattctggttctatatcaactggagtctgcatctgtaaatttttcagctggtgtcgacaattcggttaaggatgcgctggcaaaggcaaaaaccgcttctggattacagctgattctcactggctgtaaggcaaaaagaacgaagttccaacaaaccggaaaccatgctctgctagttcttgctaagagtcaagatgagttcttaagatcaactagtagcgatgaactttcttgtgaaagtcctgaggaattgcaacttaatgacgaggtcttgttagaaaggccttactacgagccattggaggaagagaatacactagaatcagaagaaaaagactctaaaagagtaaagatagatttttcttttgagccaattcttgggtccgaagaagattcagaggttaaactgttacctattgcattgaaagatgatgaaattcctccagtactgaagagtttggaccccaataatcaaccacaactagcatcactggatatggcacagcttttaatgagactaatcttcatcagagaaaatacaccctctaacagcgccatggttgaatctgaattgatggccttagttcagagaatcacgaactctaaaaatgtgaactggacaatattttcgaagagtttgtgggaaagatctttgctggaagctagtaaatcaaagactattgaaagaggcgtactgcaaatgcaggtactggttgaagatcttggaatgaatagtgtcaagaccagaatgttcagtcaatcgggagacgaacagaaatttcttaaagctctgattgatccagatgactttcaagttactcccaatgctgcgagattgaaatacatttttcagctgccgttgactccaaggtgggccctagactcacttttggctgaaaagttgatgtcaattggagtaattaagtcggctttacagatatatgaaaggctgaatctttcggtggatgctgctttgtgctatgctttaacaggtgatgaagctcaggctgaaaaaattcttgtagagcgtatagctgcccatccatccgatgcccgtgcactttcggtgttaggagatgtgaaacaatctcctgagctctggttaaagagttgggaggttggaaagtattctaaagccaaagtttctcttggaaaatactattatcaacctcctaaggattccggtttaacaaaggatttgccagctgctattggccacatttttgacgcattgacccgtaatccagttgattttgaaacttggtttttctatggttgcatgggaattgaagcatctaactggaacctcgctgctgaggcattcacaagatgtgtatccatagacaattcaagccctcaatcatggtcaaatcttgcttctgcgttaatcaaacttggtaagaataaagaagctttcagtgcattgaagagtgcaatccgaactagccaagacaagaaggtttcttcaaaaatctgggacaatttcttgatcgtggccgccaaattgagcgattggacttctgtcttactagcctcaagggaacttttaaccttgagaagcaattcgaaatccagtgaagaaattgtggatctccccgtagttgaaaagttggttcaagtactactggaatcaccctatccaggttcagaggattctatggattactttcaaaaatcgtgcgttgacttcatatgtactatgttgccacaagttatcaacaactctgacagatcttggagaattgtttctagagttgaactttggagaaagagaccttggatggcacttgagtgcaatgaaaaggcatacagggctgcggtgaacaatcctgaacttgagttcaatgaaactgcgtggaagactgctgttgatgcgtgtgctgacttggtagctgcttttgaatcattgggagagttacctggcaaacatggagccgacgatcttgtttgtaaagattggaagtataaagctaaaatgacagttcggtccctcatgtcaaagggaaaatctgcctgggaagattcagaaggctggaatgtgctacaagagctaaaaaacagtctaatgagttaa

SEQ ID NO: 4 - protein, 957 aa, Komagataella phaffii
MSALRSIHSSFIRLNIELNVGVEGFNEQTEAISLISKGQYLKVLNLPLFKKIFTDNSEIKHIIAKYTQQSSASALVVLDLVNHIIMQLSKNLESLSPEVVKTTVLILGVIFLQLFLQENFTGPSIPFENEKLPLGILRDLLKLNKEASTHILSVGGETAYELMESPILLVLSVKILEEVSGSSVSVLSKDFIDMDSETFISKCNIVTNPDVVQSSIQWWKSRAYMVFASVLTGEVSLLTLLPIKLLTPNIVSTLIDLQNKNSPENQEILVLYQLESASVNFSAGVDNSVKDALAKAKTASGLQLILTGCKAKRTKFQQTGNHALLVLAKSQDEFLRSTSSDELSCESPEELQLNDEVLLERPYYEPLEEENTLESEEKDSKRVKIDFSFEPILGSEEDSEVKLLPIALKDDEIPPVLKSLDPNNQPQLASLDMAQLLMRLIFIRENTPSNSAMVESELMALVQRITNSKNVNWTIFSKSLWERSLLEASKSKTIERGVLQMQVLVEDLGMNSVKTRMFSQSGDEQKFLKALIDPDDFQVTPNAARLKYIFQLPLTPRWALDSLLAEKLMSIGVIKSALQIYERLNLSVDAALCYALTGDEAQAEKILVERIAAHPSDARALSVLGDVKQSPELWLKSWEVGKYSKAKVSLGKYYYQPPKDSGLTKDLPAAIGHIFDALTRNPVDFETWFFYGCMGIEASNWNLAAEAFTRCVSIDNSSPQSWSNLASALIKLGKNKEAFSALKSAIRTSQDKKVSSKIWDNFLIVAAKLSDWTSVLLASRELLTLRSNSKSSEEIVDLPVVEKLVQVLLESPYPGSEDSMDYFQKSCVDFICTMLPQVINNSDRSWRIVSRVELWRKRPWMALECNEKAYRAAVNNPELEFNETAWKTAVDACADLVAAFESLGELPGKHGADDLVCKDWKYKAKMTVRSLMSKGKSAWEDSEGWNVLQELKNSLMS

SEQ ID NO: 5 - DNA, 921 bp, Komagataella phaffii
  source 1..921: mol_type = genomic DNA
atgggttgctttagattttgtctggtcataataccagccttactgtcggttgtcgcatgtttatttgcaattttctcctgtatcggatcaaccaaaaataacgagtttttgacgtctatttaccttttggaaattgatgcctccaatatatcaatttcggctctgatccctgctgccgccaatatagacatctctcctcaacaattgggtttgtcagatgtctacactcttggtatgtggggatactgcgagggtacagcttcctctgatgctcaaactgacagagatgaaattcttggtctggacaatgttgatttcaccgcttgttcaagtccaaaggcaatgtacgtttttgatcccgaaagtttcatacaggatgtgctgaatacctcggcatcactaaatcaagtgaactcatacctggacattcttggagtagacaacgttgacatctctcttcctcaggatttggtcgactacgaggacacaataagagcagtatccaagatgatctttatttgcaccataatcggtattgtgcttacattcatacaggttctcttctccattggagctttcttcagcaagggatggtcttgtgccaccactgttgtaagtatattgtccttcgtaagtcttatcattggtgctggtggtgcgactgggatgtaccgaattgtgcaaactattttcaatgataacctcggtgagtatggagtcagagcaggtctttctagaaactttttggtcttctactggcttgcggttgcattaaacctgatagccttagtattttggatcttttcaatctgctgtggatctactaggaagcgtagtaggtcgtacaccgattccgcagagaagcagccgatgatggtctatcaaccgtatgaacaggctccacagcacgtatacaaatag

SEQ ID NO: 6 - protein, 306 aa, Komagataella phaffii
MGCFRFCLVIIPALLSVVACLFAIFSCIGSTKNNEFLTSIYLLEIDASNISISALIPAAANIDISPQQLGLSDVYTLGMWGYCEGTASSDAQTDRDEILGLDNVDFTACSSPKAMYVFDPESFIQDVLNTSASLNQVNSYLDILGVDNVDISLPQDLVDYEDTIRAVSKMIFICTIIGIVLTFIQVLFSIGAFFSKGWSCATTVVSILSFVSLIIGAGGATGMYRIVQTIFNDNLGEYGVRAGLSRNFLVFYWLAVALNLIALVFWIFSICCGSTRKRSRSYTDSAEKQPMMVYQPYEQAPQHVYK

SEQ ID NO: 7 - DNA, 1263 bp, synthetic construct
  source 1..1263: mol_type = other DNA
atgaaggtgtccctgtttatctggtcctccttgatcgttggtttggtttccggtttgttgccagttgagtacccacagttcaagaagttctccaacagaaccatcatccagaacaacaactccccaggtagaaaccagtctgcttccagatttcacaagccatccaagttgtacggtgttgctcttggtggttggttggttttggagccttacatcactccatccttgttcaacgacaccgtcgaagaaactgttgacgagtacaccttgtgctacaagctgggtaagaagaaggtcaccgagttgttgactaaccactggtccactttcatcaccgagtccgacatcgtcaagatcaagaacgtcggtttgaactccgtcagaatcccaattggttactgggcctacgatttgttggaggacgacccttacattcaaggtcaggatgagttcttggcccagtgtattcactggtgtgctaagcacggtttgtccgtttggattgacttgcatggtgctccaaactctcagaacggtttcgacaactctggtagaagaggtagagctggttggcaagacgaccagagatatatcgacaagaccttggacgtcttgggtactatcgctaagagacacggtaacaagccaaacgtcatcggtatcgagattctgaacgaaccattcggtccagtcctgaacatcgacaagttgaagcagttctacgtcaagggtatcgaggtcatcagaaacaccggttactccaaggacgttgttatctccgatgctttccagggtattttccactgggacaactttttgccatccgcctccaacatcatcttggacagacatcactacgaggtgttctccgatggtcagttgagatcctcattcgagggtcacttgagaggtattgaggctttcggtagagctatcgctatcgagaagccaaccgttgttgttggtgaatggtccgctgctattactgattgtgctccatgggttaacggtgctggtagaccatctagataccacggtatggtcttggaggatggaaccattggtaactgttctggtgctaccaacattgctcaatgggccgagaagagaagagatgagttgtctaagatcctgcagacctccttgaaggcttacaacgctgctgatggttggttcttctggtgttggaaaactgactctgctttggagtgggacatggaaaagttgttggaacacggtctgttcccattggttggtgcttaa

SEQ ID NO: 8 - protein, 420 aa, Komagataella phaffii
MKVSLFIWSSLIVGLVSGLLPVEYPQFKKFSNRTIIQNNNSPGRNQSASRFHKPSKLYGVALGGWLVLEPYITPSLFNDTVEETVDEYTLCYKLGKKKVTELLTNHWSTFITESDIVKIKNVGLNSVRIPIGYWAYDLLEDDPYIQGQDEFLAQCIHWCAKHGLSVWIDLHGAPNSQNGFDNSGRRGRAGWQDDQRYIDKTLDVLGTIAKRHGNKPNVIGIEILNEPFGPVLNIDKLKQFYVKGIEVIRNTGYSKDVVISDAFQGIFHWDNFLPSASNIILDRHHYEVFSDGQLRSSFEGHLRGIEAFGRAIAIEKPTVVVGEWSAAITDCAPWVNGAGRPSRYHGMVLEDGTIGNCSGATNIAQWAEKRRDELSKILQTSLKAYNAADGWFFWCWKTDSALEWDMEKLLEHGLFPLVGA

SEQ ID NO: 9 - DNA, 1251 bp, synthetic construct
  source 1..1251: mol_type = other DNA
atgggcatgaacttgtacctgattaccttgctgttcgcctccttgtgttccgctttgactttgccaaagagagacatcatctgggactactccgacgagaagatcaagggtgttaaccttggtggttggttggtcttggagccattcattactccatccttgttcgaggctttcggtgacgatgttccagttgacgagtacagatacaccgagagattgggaaagtccttggccttggacagattgcaacaacactggtctacttggtacgaagagaaggacttccaggacattgcttcctacggtttgaacttcgtcagaatcccaatcggttactgggccttccagttgttggatgatgacccatacgttcagggtcaagaagagtacttggacaaggctttggagtggtctagaaagcacggattgaaggtctggattgacttgcatggtgctccaggttctcaaaacggtttcgacaactctggtaagagagattcctgggacttccaaaagggtgacaacgttcaggttaccttggacgtcttgaagtacatctccaagaagtacggtactgccgactactacgacgttgttatcggtatccaactgctgaacgaaccattgggtccaatcctgaacatggacaacctgagaaagttctacgctgacggttacgacttggtcagagatgtcggtaacaacttcgttgtcatccacgacgctttctaccaagaaccagaatactggggtgacgacttcacttctgctgaaggttactggaacgtcgttttggatcaccaccactaccaagttttcgacgctgaccaattgcagagatccattgacgaacacgttgaggttgcttgcaactggtctagagatgccaacaaagagtaccactggaacttgtgtggtgaatggtctgctgctttgactgactgtactccatggttgaacggtgtcggtaagggtactagatacgagggtcaattggatggttccccatggattggttcttgcgagaactctcaagacccatccaagttgtcctccgagagaatctgtgagtacagaagatacgttgaggctcagttggacgccttcttgtacggtaaatctgccggtttcatcttctggtgcttcaagactgaagcttccttggagtgggacttcaagagattggttaacgccggtattatgccacagccattggacgatagacagtacccaaaccagtgtggtttctaa

SEQ ID NO: 10 - protein, 416 aa, Komagataella phaffii
MGMNLYLITLLFASLCSALTLPKRDIIWDYSDEKIKGVNLGGWLVLEPFITPSLFEAFGDDVPVDEYRYTERLGKSLALDRLQQHWSTWYEEKDFQDIASYGLNFVRIPIGYWAFQLLDDDPYVQGQEEYLDKALEWSRKHGLKVWIDLHGAPGSQNGFDNSGKRDSWDFQKGDNVQVTLDVLKYISKKYGTADYYDVVIGIQLLNEPLGPILNMDNLRKFYADGYDLVRDVGNNFVVIHDAFYQEPEYWGDDFTSAEGYWNVVLDHHHYQVFDADQLQRSIDEHVEVACNWSRDANKEYHWNLCGEWSAALTDCTPWLNGVGKGTRYEGQLDGSPWIGSCENSQDPSKLSSERICEYRRYVEAQLDAFLYGKSAGFIFWCFKTEASLEWDFKRLVNAGIMPQPLDDRQYPNQCGF

SEQ ID NO: 11 - protein, 585 aa, Homo sapiens [sequence omitted]

SEQ ID NO: 12 - DNA, 1758 bp, synthetic construct
  source 1..1758: mol_type = other DNA
gacgctcacaagtctgaagttgcccacagattcaaggacctgggtgaagaaaacttcaaggccctggttttgatcgctttcgctcaatacttgcagcagtgtccattcgaggaccacgttaagttggttaacgaggttactgagttcgccaagacttgtgttgctgacgaatctgctgagaactgcgataagtccttgcacactttgttcggtgacaagttgtgtaccgttgccaccttgagagaaacctacggtgaaatggctgactgttgtgctaagcaagagccagaaagaaacgagtgcttcttgcagcacaaggacgacaacccaaacttgccaagattggttagaccagaggttgacgttatgtgtactgccttccacgacaacgaggaaaccttcttgaagaagtacctgtacgagatcgctagaaggcacccatacttttacgctccagagttgttgttcttcgccaagcgttacaaggctgctttcactgagtgttgtcaagctgctgacaaggccgcttgtttgttgccaaagttggacgagttgcgtgacgaaggtaaggcttcttccgctaagcagagattgaagtgtgcctccttgcaaaagttcggtgagagagcttttaaggcttgggctgttgctagactgtcccagagatttccaaaggctgagtttgctgaggtttccaagttggtcactgacttgactaaggttcacaccgaatgttgccacggtgacttgttggaatgtgctgatgatagagccgacctggccaagtacatttgtgaaaaccaggactccatctcctccaagttgaaagagtgttgcgagaagccactgttggagaagtctcactgtatcgctgaagttgagaacgacgaaatgccagctgacttgccatctttggctgctgacttcgttgaatccaaggacgtctgtaagaactacgctgaagctaaggacgtgttcctgggtatgttcttgtacgagtacgctcgtagacacccagactactccgttgtcttgttgttgagattggccaagacctacgagactaccttggaaaagtgttgtgctgctgctgacccacacgaatgttacgctaaggttttcgacgagttcaagccattggttgaggaaccacagaacctgatcaagcagaactgtgagttgttcgagcagctgggtgagtacaagttccagaacgctttgttggtcagatacaccaagaaggtcccacaggtttccactcctaccttggttgaagtctccagaaaccttggtaaggtcggttccaagtgttgtaagcacccagaggctaagagaatgccatgtgctgaagattacttgtccgtcgtcttgaaccagttgtgcgtcttgcacgaaaagactccagtttccgacagagttaccaagtgctgtactgagtccttggtcaaccgtagaccatgtttctctgctttggaagtcgacgagacttacgtcccaaaagagttcaacgctgagactttcactttccacgctgacatctgtaccctgtccgaaaaagagagacagatcaagaagcagactgccttggtcgaattggtcaagcacaagccaaaggctaccaaagagcagttgaaggctgttatggatgacttcgctgctttcgttgagaagtgttgcaaggctgacgacaaagagacttgtttcgctgaagagggtaagaagttggttgctgcttcccaagctgctttgggtctgtaa

SEQ ID NO: 13 - DNA, 20 bp, synthetic construct
  source 1..20: mol_type = other DNA
ggtgctggaagagactgcag

SEQ ID NO: 14 - DNA, 20 bp, synthetic construct
  source 1..20: mol_type = other DNA
gatgggcagtaaggtcaaca

SEQ ID NO: 15 - DNA, 20 bp, synthetic construct
  source 1..20: mol_type = other DNA
ggtatgtggggatactgcga

SEQ ID NO: 16 - DNA, 4128 bp, synthetic construct
  source 1..4128: mol_type = other DNA
atggacaagaagtactccatcggtctggacatcggaactaactctgttggttgggctgttatcaccgacgagtacaaggttccatccaagaagttcaaggtcctgggtaacactgacagacactccatcaagaagaacttgatcggagccttgttgttcgactctggtgaaactgctgaggccaccagattgaagagaactgccagaagaagatacaccagacgtaagaacagaatctgctacctgcaagagattttctccaacgagatggccaaggtcgacgactcatttttccacagattggaagagtccttcctggtcgaagaggataagaagcacgagagacacccaatcttcggtaacatcgttgacgaggttgcttaccacgagaagtacccaactatctaccacctgagaaagaagttggttgactccactgacaaggccgacttgagattgatctacttggctttggcccacatgattaagttcagaggtcacttcttgatcgagggtgacttgaacccagacaactctgacgttgacaagctgttcatccagttggtccagacctacaaccagctgttcgaagagaaccctattaacgcttctggtgttgacgctaaggctatcttgtctgccagattgtccaagtccagaagattggagaacctgatcgctcaattgccaggtgagaagaagaacggtttgttcggtaacttgattgccctgtccttgggtttgaccccaaacttcaagtccaacttcgatttggctgaggacgccaagttgcagttgtctaaggatacttacgacgacgacctggacaacttgttggctcaaattggtgaccagtacgccgacttgtttttggctgctaagaacttgtccgacgccatcttgttgtccgacatcttgagagttaacaccgagatcactaaggctccattgtccgcttccatgatcaagagatacgacgaacaccaccaggacttgactttgttgaaggccttggttagacagcagctgcctgagaagtacaaagaaatcttcttcgatcagtccaagaacggctacgctggttacattgatggtggtgcttctcaagaagagttctacaagttcatcaagcccatcttggagaagatggacggtactgaagagttgctggtcaagttgaacagagaggacttgctgagaaagcagagaaccttcgacaacggttccattccacaccagattcacttgggtgagttgcacgctatcttgcgtagacaagaggacttctacccattcctgaaggacaacagagagaagatcgaaaagatcctgaccttcagaatcccctactacgttggtccattggctagaggtaactctagattcgcttggatgaccagaaagtccgaagagactatcaccccatggaacttcgaagaggttgttgacaagggtgcttccgctcaatccttcatcgagagaatgactaacttcgacaagaacctgccaaacgagaaggtcttgccaaagcactctttgctgtacgagtacttcaccgtctacaacgagctgactaaggtcaagtacgttaccgagggtatgagaaagccagctttcttgtctggtgagcaaaagaaggctatcgtcgacttgctgttcaagaccaacagaaaggttaccgtcaagcagctgaaagaggactacttcaagaaaatcgagtgcttcgactccgtcgagatttccggtgttgaggatagattcaacgcctccttgggaacctaccacgacttgttgaagatcatcaaggacaaggatttcttggacaacgaggaaaacgaggacattttggaggacatcgtcttgaccttgactctgttcgaggacagagagatgattgaggaaagacttaagacttacgcccacctgttcgacgacaaggttatgaagcaactgaagaggcgtagatacaccggttggggtagattgtctagaaagctgatcaacggtatcagagacaagcagtccggtaagactatcctggactttttgaagtccgacggtttcgccaaccgtaacttcatgcaattgatccacgacgactccctgactttcaaagaggacattcagaaggctcaggtttccggtcaaggtgattccttgcatgagcacattgctaacttggctggttccccagctatcaagaagggtatcttgcagaccgttaaggttgtcgacgagttggttaaggttatgggtagacacaagcccgagaacatcgttatcgaaatggctagagagaaccagactacccagaagggtcaaaagaactccagagaaagaatgaagaggatcgaagagggtatcaaagagctgggttcccagattttgaaagagcacccagttgagaacacccagctgcagaacgaaaagctgtacttgtactacttgcagaacggtagagacatgtacgtcgaccaagagctggacattaacagactgtctgactacgacgttgaccacatcgttccacagtcttttctgaaggacgactccatcgacaacaaggtcttgactagatccgataagaacaggggtaagtccgacaacgttccttctgaagaggtcgtcaagaagatgaagaactactggcgtcagttgctgaacgccaagctgattactcagaggaagttcgacaacttgactaaggctgagagaggtggtttgtccgaattggataaggccggtttcatcaagagacagctggtcgagactagacagatcacaaagcacgttgcccaaatcttggactccagaatgaacactaagtacgacgagaacgacaagttgatccgtgaggttaaggtcatcaccctgaagtccaagttggtgtccgatttcagaaaggacttccaattctacaaggtccgtgagatcaacaactaccaccatgctcacgacgcttacttgaacgctgttgttggtactgccctgatcaaaaagtaccctaagctggaatccgagttcgtctacggtgactacaaggtttacgacgtcagaaagatgatcgccaagtccgaacaagagatcggtaaggctactgccaagtactttttctactccaacatcatgaactttttcaagactgagatcaccctggccaacggtgagatcagaaaaagaccactgatcgagactaacggtgaaaccggtgaaatcgtttgggacaagggtagagacttcgccaccgttagaaaggttttgtccatgccacaggtcaacatcgtcaaaaagactgaggttcagaccggtggtttctccaaagagtccatcttgcctaagagaaactccgacaagctgatcgccagaaagaaggattgggacccaaagaagtacggcggtttcgattctccaactgttgcctactccgttttggttgttgctaaggtcgaaaagggcaagtctaagaagctgaagtccgtcaaagaactgctgggtatcactatcatggaaaggtccagctttgagaagaacccaatcgactttttggaggccaagggttacaaagaggtgaagaaggacctgattatcaagctgccaaagtactccctgttcgagttggaaaacggtagaaagagaatgttggcttccgccggtgaattgcaaaagggtaacgaattggctctgccatccaagtacgtcaactttctgtacttggcctctcactacgagaagttgaagggttctccagaggacaacgaacagaagcagttgttcgttgagcagcacaagcactacttggacgagatcattgagcagatttccgagttctccaagcgtgttattttggctgatgccaacctggataaggtcttgtccgcctacaacaagcacagagataagccaattagagagcaggctgagaacatcatccacttgttcactttgactaacctgggtgccccagctgcttttaagtacttcgacactaccatcgacagaaagagatacacctccaccaaagaggttttggacgctactttgatccaccagtccatcactggtctgtacgaaaccagaattgacttgtcccagcttggtggtgaccctaagaagaagagaaaggtctaa

SEQ ID NO: 17 - protein, 1375 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDPKKKRKV

SEQ ID NO: 18 - DNA, 267 bp, synthetic construct
  source 1..267: mol_type = other DNA
atgagattcccctccatcttcaccgctgttttgttcgctgcttcttctgctttggctgctccagttaacactactactgaggacgagactgctcagattccagctgaagctgttattggttactccgacttggaaggtgacttcgacgttgctgttttgccattctccaactccaccaacaacggtctgttgttcatcaacaccactatcgcttccattgccgctaaagaagaaggcgtttccttggagaagagagaggctgaagct

SEQ ID NO: 19 - DNA, 549 bp, synthetic construct
  source 1..549: mol_type = other DNA
gttgtagttttaatatagtttgagtatgagatggaactcagaacgaaggaattatcaccagtttatatattctgaggaaagggtgtgtcctaaattggacagtcacgatggcaataaacgctcagccaatcagaatgcaggagccataaattgttgtattattgctgcaagatttatgtgggttcacattccactgaatggttttcactgtagaattggtgtcctagttgttatgtttcgagatgttttcaagaaaaactaaaatgcacaaactgaccaataatgtgccgtcgcgcttggtacaaacgtcaggattgccaccacttttttcgcactctggtacaaaagttcgcacttcccactcgtatgtaacgaaaaacagagcagtctatccagaacgagacaaattagcgcgtactgtcccattccataaggtatcataggaaacgagagtcctccccccatcacgtatatataaacacactgatatcccacatccgcttgtcaccaaactaatacatccagttcaagttacctaaacaaatcaaa

SEQ ID NO: 20 - DNA, 399 bp, synthetic construct
  source 1..399: mol_type = other DNA
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 21 - DNA, 810 bp, synthetic construct
  source 1..810: mol_type = other DNA
atgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctggctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa